(19) United States
(12) Patent Application Publication
(10) Pub. No.: US 2025/0066430 A1
(43) Pub. Date: Feb. 27, 2025

(54) INSECTICIDAL PROTEIN AND USES THEREOF

(71) Applicant: DCM SHRIRAM LIMITED, New Delhi (IN)

(72) Inventors: Santosh Kumar DODDA, Hyderabad, Telangana (IN); Lavanya SEELAM, Hyderabad, Telangana (IN); Dwarkesh Singh PARIHAR, Hyderabad, Telangana (IN); Paresh Kumar VERMA, Hyderabad, Telangana (IN)

(73) Assignee: DCM SHRIRAM LIMITED, New Delhi (IN)

(21) Appl. No.: 18/725,034

(22) PCT Filed: Jan. 5, 2023
(86) PCT No.: PCT/IN2023/050011

§ 371 Date: Jun. 27, 2024

(30) Foreign Application Priority Data
Jan. 6, 2022 (IN) 202211000823

(51) Int. Cl. C07K 14/32; A01N 63/50; A01P 7/04; C12N 15/82

(52) U.S. Cl. CPC C07K 14/32; A01N 63/50; A01P 7/04; C12N 15/8286

(57) ABSTRACT

Disclosed herein are novel vegetative insecticidal proteins (Vip) from Bacillus thuringiensis, nucleotide sequences encoding the proteins, and primers for the identification of the genes encoding the said proteins active against insect pests. The Vip proteins disclosed herein exhibits activity against a wide range of insect pests including, but not limited to the insect pests belonging to Lepidoptera. The nucleotide sequences encoding the said Vip proteins can be used to transform various prokaryotic and eukaryotic organisms including plants to express one or more Vip proteins. These recombinant organisms can be used to control a wide range of insect pests including but not limited to lepidopteran insects.

### REFERENCE TO SEQUENCE LISTING SUBMITTED ELECTRONICALLY

An official copy of the Sequence Listing a file named “PD0453781N-SC_SEQUENCE LISTING.xml” created on 5 Jan. 2023, having a size of 36 kb filed electronically concurrently with the Specification is part of the Specification.

### FIELD

The present disclosure is related to a novel pesticidal toxin protein isolated from Bacillus thuringiensis (Bt). The protein disclosed herein exhibits activity against a wide range of insect pests including, but not limited to the insect pests belonging to Lepidoptera. The present disclosure also relates to the DNA sequence(s) encoding the said protein. The DNA sequence encoding the pesticidal toxin protein can be used to transform various prokaryotic and eukaryotic organisms including plants to express the pesticidal toxin protein. These recombinant organisms can be used to control lepidopteran insects in various environments.

### BACKGROUND

Plant health is increasingly under threat due to climate change and human activities. Climate change has altered ecosystems, reducing biodiversity and creating new niches where pests can thrive. Recent study shows that warmer climate will increase the metabolic rate of insects, which is the rate at which they digest what they eat. Thus due to climate change insects are going to be hungrier and their population will increase manifold causing more crop losses. Also, the rise in temperatures will lead to an increase in population of these pests.

Pests and pathogens that damage crops are worldwide problem in agricultural sector. Agriculture crop productivity has been severely affected by various pests resulting in economic hardships to farmers and nutritional deprivation for local populations in many parts of the world. Food and Agriculture Organization (FAO) estimates that about 40% of food crops are lost annually due to plant pests and diseases. Each year, plant diseases cost the global economy around $220 billion, and invasive insects around US$70 billion. This leaves millions of people without enough food to eat and seriously damages agriculture which is a primary source of income for rural poor communities.

Plant pests and diseases are often impossible to eradicate once they have established themselves and managing them is time consuming and expensive. Therefore, protecting plants from pests and diseases is an important aspect in order to reduce crop damages thereby increasing the crop productivity. Chemical pesticides are being used to control the plant pests, however, the excessive use of chemicals in agriculture has led to a multitude of effects, including increased residues in plants, insect resistance, and contamination of soil, water, and air. Disturbances in the food chain due to the accumulation of pesticide residues have a significant impact on ecology. Bio-pesticides have also been used in agriculture as an alternative solution to chemical pesticides. However, these methods are not enough and there is a substantial interest in developing effective alternative methods.

Control of various pests through the use of biological molecules has been possible in only a limited number of cases. The δ endotoxins from Bacillus thuringiensis (Bt)—a gram-positive spore forming microorganism are the best known examples of biological molecules. Varieties of Bt are known that produce more than 25 different but related δ-endotoxins. Bt strains produce δ-endotoxins during sporulation. The majority of δ-endotoxins made by Bt are toxic to larvae of certain insects in the orders Lepidoptera, Diptera and Coleoptera. Some of these δ-endotoxins have useful insecticidal activities against different insect pests. However, use of the δ-endotoxins is limited because they are active against only a very few of the many insect pests. The limited specificity of the Bt endotoxins is dependent, at least in part, on both the activation of the toxin in the insect gut and its ability to bind to specific receptors present on the insect's midgut epithelial cells.

Among the factors which prevent activity of a particular δ-endotoxin against a specific insect is the lack of appropriate receptors in the insect gut or lack of affinity of the δ-endotoxin for the receptors which may be present, thus resulting in no binding of the delta-endotoxin to the brush border membranes. Therefore, the ability to control a specific insect pest using δ-endotoxins at present depends on the ability to find an appropriate δ-endotoxin with the desired range of activity. However, these molecules had a limited period of successful use in many crop-pathogen systems due to the rapid evolution of pests to overcome resistance genes.

Another family of insecticidal proteins produced by Bacillus species during the vegetative stage of growth (vegetative insecticidal proteins (Vip) has also been identified.

Therefore, scientists must constantly be in search of new molecules, compositions or methods with which crops can be protected against plant pests. It has been found in the present invention a novel insecticidal toxin protein which can be used to control plant insect pests. The toxin protein and the nucleotide sequence encoding the same of the current invention are distinct from the toxin protein and the nucleotide sequence known in the art.

### BRIEF SUMMARY

The present invention relates to materials and methods useful in the control of plant insect pests. In one embodiment, the present invention provides novel Bt toxins useful for the control of insect pests. In a preferred embodiment, these insect pests includes but not limited to lepidopterans and coleopterans. The toxin protein of the present invention is vegetative insecticidal proteins (Vip) toxin in particular Vip 3 proteins.

The present invention further relates to nucleotide sequences which encode the insecticidal proteins disclosed in the present invention. The invention further provides nucleotide sequences and methods useful in the identification and characterization of nucleotide sequence(s) which encode the insecticidal proteins.

The present invention specifically relates to an insecticidal protein having an amino acid sequence selected from the group consisting of SEQ ID NO: 1, SEQ ID NO: 7 and SEQ ID NO: 11, when expressed in a plant, results in an insecticidal activity.

The present invention also relates to a recombinant nucleic acid molecule comprising a nucleotide sequence encoding the protein of the present invention, wherein said nucleotide sequence selected from the group consisting of SEQ ID NO: 2, SEQ ID NO: 5, SEQ ID NO: 6, SEQ ID NO: 8, SEQ ID NO: 9, SEQ ID NO: 10, SEQ ID NO: 12, SEQ ID NO: 13 and SEQ ID NO: 14.

Further, the present invention relates to an expression cassette, vector, host cells, and or microorganism comprising the nucleotide sequences disclosed in the present invention.

The present invention also relates to a method for producing a plant exhibiting an insecticidal activity, wherein said method comprises at least the step of introducing the nucleic acid molecule as disclosed in the present invention. The present invention further relates to a transgenic plant comprising a recombinant nucleic acid molecule comprising a nucleotide sequence encoding the protein(s) disclosed herein, wherein said plant exhibits an insecticidal activity.

The present invention further relates to a recombinant microorganism comprising a recombinant nucleotide sequence which encodes the insecticidal protein disclosed in the present invention and a composition comprising the recombinant microorganisms of the present invention.

The present invention further relates to use of the insecticidal proteins, the recombinant nucleic acid molecules as disclosed in the present invention for production of insecticidal composition.

The present invention further relates to use of the recombinant nucleic acid molecule, the expression cassette and the vector as disclosed herein for production of insect resistant transgenic plants.

The present invention further relates to use of the recombinant microorganism and the composition disclosed herein for the control of insect pests.

### BRIEF DESCRIPTION OF THE SEQUENCES

SEQ ID NO: 1 shows amino acid sequence of the novel Vip protein-1 as disclosed herein.

SEQ ID NO: 2 shows Native DNA sequence encoding Vip protein-1 having the amino acid sequence as set forth in SEQ ID NO: 1.

SEQ ID NO: 3 shows DNA sequence of forward degenerate primer.

SEQ ID NO: 4 shows DNA sequence of reverse degenerate primer.

SEQ ID NO: 5 shows modified DNA sequence encoding Vip protein-1 having the amino acid sequence as set forth in SEQ ID NO: 1.

SEQ ID NO: 6 shows modified DNA sequence encoding Vip protein-1 having the amino acid sequence as set forth in SEQ ID NO: 1.

SEQ ID NO: 7 shows amino acid sequence of the novel Vip protein-2.

SEQ ID NO: 8 shows Native DNA sequence encoding a novel Vip protein-2 having the amino acid sequence as set forth in SEQ ID NO: 7.

SEQ ID NO: 9 shows modified DNA sequence encoding a novel Vip protein-2 having the amino acid sequence as set forth in SEQ ID NO: 7.

SEQ ID NO: 10 shows modified DNA sequence encoding a novel Vip protein-2 having the amino acid sequence as set forth in SEQ ID NO: 7.

SEQ ID NO: 11 shows amino acid sequence of the novel Vip protein-3.

SEQ ID NO: 12 shows Native DNA sequence encoding a novel Vip protein-3 having the amino acid sequence as set forth in SEQ ID NO: 11.

SEQ ID NO: 13 shows modified DNA sequence encoding a novel Vip protein-3 having the amino acid sequence as set forth in SEQ ID NO: 11.

SEQ ID NO: 14 shows modified DNA sequence encoding a novel Vip protein-3 having the amino acid sequence as set forth in SEQ ID NO: 11.

### BRIEF DESCRIPTION OF THE FIGURES

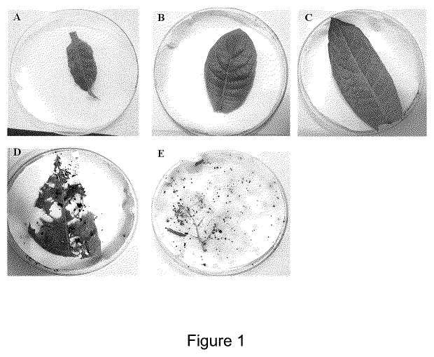

FIG. 1 shows detached leaf bioassay against Spodoptera litura.
A: Transgenic tobacco leaf expressing the Vip protein-1; B: Transgenic tobacco leaf expressing the Vip protein-2; C: Transgenic tobacco leaf expressing the Vip protein-3; D: Non-transgenic control sample-1; and E: Non-transgenic control sample-2.

### DETAILED DESCRIPTION

The detailed description provided herein is to assist person skilled in the art in practicing the present invention and should not be construed to unduly limit scope of the invention as modifications and variations in the embodiments discussed herein may be made by those of person skilled in the art without departing from the spirit or scope of the invention. The present inventions will be described more fully hereinafter with reference to the accompanying drawings and/or sequence listing, in which some, but not all embodiments of the inventions are shown and/or described. The invention may be embodied in many different forms and should not be construed as limited to the embodiments set forth herein.

Although specific terms are employed herein, they are used in a generic and descriptive sense only and not for purposes of limitation. The following definitions are provided to facilitate understanding of the embodiments.

It must be noted that, as used in the specification and the appended claims, the singular forms “a,” “an” and “the” are used herein to refer to one or more than one (i.e., to at least one) of the grammatical object of the article unless the context clearly dictates otherwise. Thus, for example, reference to “a probe” means that more than one such probe can be present in the composition. Similarly, reference to “an element” means one or more element.

Throughout the specification the word “comprises” or “comprising,” will be understood to imply the inclusion of a stated element, integer or step, or group of elements, integers or steps, but not the exclusion of any other element, integer or step, or group of elements, integers or steps.

Units, prefixes, and symbols may be denoted in their SI accepted form. Unless otherwise indicated, nucleic acids are written left to right in 5′ to 3′ orientation and amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The above-defined terms are more fully defined by reference to the specification as a whole.

The term “nucleic acid” (DNA and RNA) typically refers to large polynucleotides. The term “nucleic acid” and “nucleotide sequence” are used interchangeably herein. It includes reference to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to that of naturally occurring nucleotides. Nucleotides are the subunit that is polymerized (connected into a long chain) to make nucleic acids (DNA and RNA). Nucleotides consist of three smaller components a ribose sugar, a nitrogenous base, and phosphate group(s).

A “polynucleotide” means single, parallel or anti-parallel strands of a nucleic acid. Thus, a polynucleotide may be either a single-stranded or a double-stranded nucleic acid.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

In some embodiments the non-genomic nucleic acid molecule is a cDNA. In some embodiments the non-genomic nucleic acid molecule is a synthetic nucleotide sequence. Codon-optimized nucleotide sequences may be prepared for any organism of interest using methods known in the art. Optimized nucleotide sequences find use in increasing expression of a pesticidal protein in a plant, plant cell, tissue or any part thereof for example monocot and dicot plants such as, rice, tomato, and cotton plant.

The terms “DNA construct”, “nucleotide constructs”, and “DNA expression cassette” are used interchangeably herein and is not intended to limit the embodiments to nucleotide constructs comprising DNA. Those of ordinary skill in the art will recognize that nucleotide constructs particularly polynucleotides and oligonucleotides composed of ribonucleotides and combinations of ribonucleotides and deoxyribonucleotides may also be employed in the methods disclosed herein.

A “recombinant” nucleic acid molecule or DNA or polynucleotide is used herein to refer to a nucleic acid molecule or DNA polynucleotide that has been altered or produced by the hand of man and is in a recombinant bacterial or plant host cell. For example, a recombinant polynucleotide may be a polynucleotide isolated from a genome, a cDNA produced by the reverse transcription of an RNA, a synthetic nucleic acid molecule or an artificial combination of two otherwise separated segments of sequence, e.g., by chemical synthesis or by the manipulation of isolated segments of polynucleotides by genetic engineering techniques.

The term “Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

Optimal alignment of sequences for comparison may be conducted by computerized implementations of algorithms known in the art (GAP, BESTFIT, BLAST, PASTA, and TFASTA in the Wisconsin Genetics Software Package, Genetics Computer Group (GCG), 575 Science Dr., Madison, WI), or by inspection.

“Percentage of sequence identity,” as used herein, is determined by comparing two optimally aligned sequences over a comparison window, where the fragment of the polynucleotide or amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Optimal alignment of sequences for comparison may be conducted by computerized implementations of algorithms known in the art (GAP, BESTFIT, BLAST, PASTA, and TFASTA in the Wisconsin Genetics Software Package, Genetics Computer Group (GCG), 575 Science Dr., Madison, WI), or by inspection.

The term “substantial sequence identity” between nucleotide sequences used herein refers to polynucleotide comprising a sequence that has at least 65% sequence identity, preferably at least 69% to 77% sequence identity compared to the reference sequence.

As used herein, “heterologous DNA coding sequence” or “heterologous nucleic acid” or “heterologous polynucleotide” means any coding sequence other than the one that naturally encodes the Vip protein, or any homolog of the Vip protein.

As used herein, “coding region” refers to that portion of a gene, a DNA or a nucleotide sequence which codes for a protein. The term “non-coding region” refers to that portion of a gene, a DNA or a nucleotide sequence that is not a coding region.

As used herein, the terms “encoding” or “encoded” when used in the context of a specified nucleic acid mean that the nucleic acid comprises the requisite information to direct translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

The terms “polypeptide,” “peptide,” and “protein” are used interchangeably herein to refer to a polymer composed of amino acid residues related naturally occurring structural variants, and synthetic non-naturally occurring analogs thereof linked via peptide bonds. Synthetic polypeptides can be synthesized, for example, using an automated polypeptide synthesizer. The terms apply to amino acid polymers in which one or more amino acid residues is an artificial chemical analogue of a corresponding naturally occurring amino acid, as well as to naturally occurring amino acid polymers. The terms “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass known analogues of natural amino acids that can function in a similar manner as naturally occurring amino acids.

The term “protein” typically refers to large polypeptides. The term “peptide” typically refers to short polypeptides. However, the term “polypeptide” is used herein to refer to any amino acid polymer comprised of two or more amino acid residues linked via peptide bonds.

As used herein, “expression cassette” means a genetic module comprising a gene and the regulatory regions necessary for its expression, which may be incorporated into a vector.

A “vector” is a composition of matter which comprises nucleic acid molecule and which can be used to deliver the isolated nucleic acid to the interior of a cell. Numerous vectors are known in the art including, but not limited to, linear polynucleotides, circular polynucleotides, polynucleotides associated with ionic or amphiphilic compounds, plasmids, and viruses. Thus, the term “vector” includes an autonomously replicating plasmid or a virus. The term should also be construed to include non-plasmid and non- viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “Expression vector” refers to a vector comprising a recombinant nucleic acid comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant nucleic acid.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “Operably linked” means any linkage, irrespective of orientation or distance, between a regulatory sequence and coding sequence, where the linkage permits the regulatory sequence to control expression of the coding sequence. The term “operably linked” further means that the nucleic acid sequences being linked are contiguous and, where necessary to join two protein coding regions, contiguous and in the same reading frame. The term “operably linked” also refers to a functional linkage between a promoter and a second sequence, wherein the promoter sequence initiates and mediates transcription of the DNA sequence corresponding to the second sequence.

The term “host cell” as used herein refers to a cell which contains a vector and supports the replication and/or expression of the expression vector is intended. Host cells may be prokaryotic cells such as E. coli, or eukaryotic cells such as yeast, insect, amphibian, or mammalian cells, or monocotyledonous or dicotyledonous plant cells. An example of a monocotyledonous host cell is a rice host cell and an example of a dicotyledonous host cell is eggplant or tomato host cell. When possible, the sequence is modified to avoid predicted hairpin secondary mRNA structures.

The term “toxin” or “insecticidal protein” as used herein refers to a polypeptide showing pesticidal activity or insecticidal activity. “Bt” or “Bacillus thuringiensis” toxin is intended to include the broader class of Vegetative insecticidal proteins (Vip) toxins found in various strains of Bacillus species, which includes such toxins.

The term “probe” or “sample probe” refers to a molecule that is recognized by its complement or a particular microarray element. Examples of probes that can be investigated by this invention include, but are not limited to, DNA, RNA, oligonucleotides, oligosaccharides, polysaccharides, sugars, proteins, peptides, monoclonal antibodies, toxins, viral epitopes, hormones, hormone receptors, enzymes, enzyme substrates, cofactors, and drugs including agonists and antagonists for cell surface receptors.

As used herein, the term “complementary” or “complement” refer to the pairing of bases, purines and pyrimidines that associate through hydrogen bonding in double stranded nucleic acid. The following base pairs are complementary: guanine and cytosine; adenine and thymidine; and adenine and uracil. The terms as used herein include complete and partial complementarity.

As used herein, the term “hybridization” refers to a process in which a strand of nucleic acid joins with a complementary strand through base pairing. The conditions employed in the hybridization of two non-identical, but very similar, complementary nucleic acids vary with the degree of complementarity of the two strands and the length of the strands. Thus the term contemplates partial as well as complete hybridization. Such techniques and conditions are well known to practitioners in this field.

The terms “insecticidal activity” and “pesticidal activity” are used interchangeably herein to refer to activity of an organism or a substance (such as, for example, a protein) that can be measured by, but is not limited to, pest mortality, pest weight loss, pest repellence, and other behavioural and physical changes of a pest after feeding and exposure for an appropriate length of time. Thus, an organism or substance having pesticidal activity adversely impacts at least one measurable parameter of pest fitness. For example, “insecticidal proteins” are proteins that display insecticidal activity by themselves or in combination with other proteins.

As used herein, the term “affecting insect pests” refers to controlling changes in insect feeding, growth, and/or behaviour at any stage of development, including but not limited to killing the insect, retarding growth, preventing reproductive capability, anti-feedant activity, and the like.

As used herein, the term “pesticidally effective amount” connotes a quantity of a substance or organism that has pesticidal activity when present in the environment of a pest. For each substance or organism, the pesticidally effective amount is determined empirically for each pest affected in a specific environment. Similarly, an “insecticidally effective amount” may be used to refer to a “pesticidally effective amount” when the pest is an insect pest.

A “control plant” as referred to herein is a plant not comprising the protein and/or nucleic acid of the invention.

In case the test plant is a plant comprising the protein and/or nucleic acid of the invention.

Preferably the control plant is grown under the same conditions as the test plant comprising the protein and/or nucleic acid of the invention.

As used herein, the terms “transformed plant” and “transgenic plant” refer to a plant that comprises one or more heterologous polynucleotide within its genome. The heterologous polynucleotide(s) is stably integrated within the genome of a transgenic or transformed plant such that the polynucleotide is passed on to successive generations. The heterologous polynucleotide may be integrated into the genome alone or as part of a recombinant DNA molecule.

It is to be understood that as used herein the term “transgenic” includes any plant cell, plant cell line, callus, tissue, a plant part, or a plant, the genotype of which has been altered by the presence of one or more heterologous nucleic acid. The term includes those transgenics initially obtained using genetic transformation method known in the art as well as those created by sexual crosses, conventional breeding or asexual propagation from the initial transgenic.

The term “initial transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, the term “plant” includes whole plants, plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like and progeny thereof. Parts of transgenic plants are within the scope of the embodiments and comprise, for example, plant cells, protoplasts, tissues, callus, embryos as well as flowers, stems, fruits, leaves, and roots originating in transgenic plants or their progeny previously transformed with a DNA molecule of the embodiments and therefore consisting at least in part of transgenic cells.

One skilled in the art would know that to achieve improved expression of a Bt insecticidal protein in a plant, a nucleotide sequence encoding the Bt protein, or an active variant or fragment of the protein, would first need to be prepared. Then the nucleotide sequence encoding the protein or fragment thereof would be placed into an expression cassette that functions in plants to cause the transcription of the coding sequence into a messenger RNA that is subsequently translated in the cells of the plant such that an insecticidally effective amount of the insecticidal protein is produced within the plant tissues. One skilled in the art would also know to transform a plant cell, preferably a cotton, corn, brinjal, tomato, rice, wheat, oat, grass, forage plant, cruciferous plant, fruit tree, ornamental flower, potato, carrot, kale, Arabidopsis and tobacco plant cell and the like with the nucleotide sequence embedded within the plant functional expression cassette, and to select for cells that contain the sequence and are expressing insecticidally effective amounts of the insecticidal protein, preferably the Vip protein disclosed herein or related protein or insecticidal fragment thereof, and to produce plants from such transformed cells. One skilled in the art would know to use electroporation, infusion, ballistic methods, or Agrobacterium tumefaciens mediated methods and the like for introducing the nucleotide sequences of the present invention or modifications thereof into a plant cell.

The term “variant or modified”, with reference to nucleotide sequences, is intended to refer to nucleotide sequences which encode the same toxins or which encode equivalent toxins having similar insecticidal activity, the term “equivalent toxin” referring to a toxin exhibiting the same, essentially the same, or improved biological activity against the target pests as the claimed native or referent toxin. A variant or modified nucleotide sequence intended for use in dicot plants would encode substantially the same amino acid sequence as the native coding sequence, i.e., the coding sequence found in nature, but would comprise a total combined GC composition from about 49 to about 58 percent, and would utilize substantially the codon preference and codon usage frequency determined by compiling such preference and usage frequencies from a consortium of coding sequences derived from one or more individual dicot plant species intended to be transformed with the variant or modified nucleotide sequence. A variant or modified nucleotide sequence intended for use in a monocot plant would also encode substantially the same amino acid sequence as the native coding sequence, but would comprise a total combined GC composition from about 52 to about 59 percent, and would also utilize substantially the codon preference and codon usage frequency determined by compiling such preference and usage frequencies from a consortium of coding sequences derived from one or more individual monocot plant species intended to be transformed with the variant or modified nucleotide sequence. A variant or modified nucleotide sequence intended for use in a leguminous plant would also encode substantially the same amino acid sequence as the native coding sequence, but would comprise a total combined GC composition from about 52 to about 59 percent, and would also utilize substantially the codon preference and codon usage frequency determined by compiling such preference and usage frequencies from a consortium of coding sequences derived from one or more individual leguminous plant species intended to be transformed with the variant or modified nucleotide sequence. Codon usage frequency is intended to refer to the number of times, on average, that a particular codon is used in a coding sequence. For a particular plant species, a codon that is intended to cause the incorporation of a particular amino acid into a nascent amino acid sequence will be utilized on average with some relative fixed frequency. For amino acids that utilize only two codons, this frequency is generally about fifty-fifty, i.e., each codon being used about half the time, unless one of the codons utilizes a substantially greater number of purines or pyrimidines that are not typically representative of the GC content of the particular plant species.

For Bacillus species, for example, coding sequences generally are from about 60 to about 70 percent AT. Codon usage in Bacillus species is biased toward the use of codons that are enriched for the presence of A or T in a particular codon. Therefore, codons that primarily utilize G or C are used in a native and/or naturally occurring Bacillus coding sequence with much less frequency than codons that contain A's or T's. Therefore, when producing a variant or modified nucleotide sequence intended for use in a particular plant, monocot, dicot or leguminous, it is important to ensure that appropriate attention is given to the use of codons that are not particularly enriched with A's and T's where possible, and to avoid the incorporation of suspected polyadenylation sequences.

As used herein, “synthetic coding sequences” or “non-naturally occurring coding sequences” encoding the B. thuringiensis Vip proteins as disclosed herein or homologs or derivatives thereof as insecticidal toxins of the present invention are those prepared in a manner involving any sort of genetic isolation or manipulation. This includes isolation of the coding sequence from its naturally occurring state, manipulation of the coding sequence as by modification of the nucleotide coding sequence as disclosed herein, chemical synthesis of all or part of a coding sequence using phosphoramidite chemistry and the like, or site-specific mutagenesis, truncation of the coding sequence or any other manipulative or isolative method so that the amino acid sequence encoded by the non-naturally occurring coding sequence encodes substantially the same insecticidal protein as the native coding sequence and furthermore exhibits substantially the same or an improved level of insecticidal bioactivity as the native insecticidal toxin protein.

As used herein, the phrase “percentage of sequence identity” is determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base' or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. A first nucleotide sequence when observed in the 5′ to 3′ direction is said to be a “complement” of a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every 445 nucleotide of one of the sequences read 5′ to 3′ is complementary to every nucleotide of the other sequence when read 3′ to 5′. A nucleotide sequence that is identical at every position when read 5′ to 3′ in comparison to a reference nucleotide sequence read 5′ to 3′ is said to be identical to the reference sequence and vice-versa. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

As used herein, “substantial homology”, with reference to nucleotide sequences, refers to nucleotide sequences that hybridize under stringent conditions to the Vip coding sequence as set forth in SEQ ID NO: 2 or complements thereof. Sequences that hybridize under stringent conditions to SEQ ID NO: 2 or complements thereof. Such homologous sequences are from about 67% identical, to about 70% identical, to about 80% identical, to about 85% identical, to about 90% identical, to about 95% identical, to about 99% identical or greater to the referent nucleotide sequence as set forth in SEQ ID NO: 2 or the complement thereof, wherein the nucleotide sequences encodes the Vip protein disclosed herein. In addition, nucleotide sequences that encode insecticidal proteins, that hybridize under stringent conditions to SEQ ID NO:2 are also envisioned to exhibit substantial homology with referent nucleotide sequences that hybridize under stringent conditions to the Vip coding sequence as set forth in SEQ ID NO:2 or complements thereof. Such nucleotide sequences are referred to herein as homologs of SEQ ID NO: 2 and the like.

With reference to polypeptide sequences, the term “substantial homology” refers to polypeptides that are about 70% homologous to, about 80% homologous to, about 86% homologous to, about 90% homologous to, about 95% homologous to, about 99% homologous to, a referent polypeptide sequence. More specifically, the inventors envision substantial homologues to be about 81, 82, 83, 84, 85, 86, 87, 88, 89, 90, 91, 92, 93, 94, 95, 96, 97, 98, and 99 percent homologous to the referent polypeptide sequence as set forth herein in SEQ ID NO: 1.

With reference to the proteins of the instant application, the terms “variant amino acid sequence”, or “amino acid sequence variant”, or “modified amino acid sequence variant” are intended to refer to amino acid sequences that are substantially equivalent to the amino acid sequences of the present invention. For example, a protein produced by the introduction of a restriction site for convenience of molecular manipulations into a coding sequence of the present invention that results in the addition or subtraction of one or more codons without otherwise (1) disrupting the native coding sequence, (2) disrupting the native open reading frame, and (3) disrupting the insecticidal biological activity of the protein, would constitute (a) a variant amino acid sequence compared to the native insecticidal toxin, (b) an amino acid sequence variant compared to the native insecticidal toxin, or (c) a modified amino acid sequence variant compared to the native insecticidal toxin. One skilled in the art would recognize that there are other types of modifications that can be made to the amino acid sequence of the present invention without disrupting the biological activity of the protein. Insertions, deletions, and substitutions are within the scope of the present disclosure to the extent that the resulting amino acid sequence variant exhibits insecticidal activity no less than that of the native insecticidal protein. Chimeras of the proteins disclosed herein, fusions of the proteins or parts of the proteins disclosed herein, and permuteins of the proteins disclosed herein are specifically contemplated.

Bacillus thuringiensis insecticidal Vip protein and Vip gene Bacillus thuringiensis (Bt) is a Gram-positive bacterium that produces insecticidal proteins as crystal inclusions during its sporulation phase of growth, known as Cry or Cyt toxins, which have been proven to be effective against important crop pests and also against mosquitoes. Bt also produces Vegetative insecticidal proteins (Vip) which are secretable proteins and do not share sequence homology with known Cry proteins and display insecticidal activity against a wide variety of lepidopterans and coleopterans and some sap-sucking insect pests.

Vip proteins are divided into four families according to their amino acid identity. The Vip1 and Vip2 proteins act as binary toxins and are toxic to some members of the Coleoptera and Hemiptera. The Vip1 component is thought to bind to receptors in the membrane of the insect midgut, and the Vip2 component enters the cell, where it displays its ADP-ribosyltransferase activity against actin, preventing microfilament formation. Vip3 has no sequence similarity to Vip1 or Vip2 and is toxic to a wide variety of members of the Lepidoptera. Its mode of action has been shown to resemble that of the Cry proteins in terms of proteolytic activation, binding to the midgut epithelial membrane, and pore formation, although Vip3A proteins do not share binding sites with Cry proteins.

The present invention encompasses the pesticidal protein disclosed herein as well as components and fragments thereof. That is, it is recognized that component protomers, polypeptides or fragments of the proteins may be produced which retain pesticidal activity. These fragments include truncated sequences, as well as N-terminal, C-terminal, internal and internally deleted amino acid sequences of the proteins.

The present invention also encompasses any deletions, insertions, and substitutions of the protein sequence which does not produce radical changes in the characteristics of the pesticidal protein. Person skilled in the art will appreciate that the effect of such deletions, insertions, and substitutions of the protein will be evaluated by routine screening assays. The proteins described herein as well as components and fragments thereof may be used alone or in combination. That is, several proteins may be used to control different insect pests.

The pesticidal protein of the invention can be used in combination with Bt endotoxins or other insecticidal proteins to increase insect target range. Furthermore, the use of the Vip protein of the present invention in combination with Bt δ-endotoxins or other insecticidal principles of a distinct nature has particular utility for the prevention and/or management of insect resistance. Other insecticidal principles include protease inhibitors (both serine and cysteine types), lectins, α-amylase and peroxidase.

In one of the preferred embodiment encompasses expression of the VIP as disclosed herein in a transgenic plant alone or in combination with one or more Bt δ-endotoxins. This co-expression of more than one insecticidal principle in the same transgenic plant can be achieved by genetic transformation and/or conventional breeding method. One skilled in the would know how to perform these method to archive the desired expression of the Vip protein as described herein in combination with one or more Bt δ-endotoxins using these methods.

For example, a transgenic plant expressing the Vip protein as disclosed herein in combination with one or more Bt δ-endotoxin or any other insecticidal protein can be obtained by crossing a transgenic plant expressing the Vip as described herein obtained using genetic transformation method or any other method known in the art with a second plant expressing one or more Bt δ-endotoxin or any other insecticidal protein obtained using genetic transformation method or any other method known in the art.

The present invention also encompasses nucleotide sequences from organisms other than Bacillus thuringiensis, where the nucleotide sequences are isolatable by hybridization with the nucleotide sequences of the present invention. Proteins encoded by such nucleotide sequences can be tested for pesticidal activity. The invention also encompasses the proteins encoded by the nucleotide sequences. Furthermore, the invention encompasses proteins obtained from organisms other than Bacillus thuringiensis wherein the protein cross-reacts with antibodies raised against the proteins of the invention. Again the isolated proteins can be assayed for pesticidal activity by the methods disclosed herein or others well-known in the art.

Once the nucleotide sequences encoding the pesticidal proteins of the invention have been isolated, they can be manipulated and used to express the protein in a variety of hosts including other organisms, including microorganisms and plants.

The nucleotide sequence encoding of the Vip of the present discloser can be optimized for enhanced expression in plants. For example the nucleotide sequences can be synthesized utilizing plant preferred codons. That is the preferred codon for a particular plant such as the monocot preferred codons, the dicot preferred codons and/or the leguminous plant preferred codons. Synthetic genes can also be made based on the distribution of codons a particular host uses for a particular amino acid. In this manner, the nucleotide sequences can be optimized for expression in any plant. It is recognized that all or any part of the gene sequence may be optimized or synthetic. That is, synthetic or partially optimized sequences may also be used.

In this manner, the nucleotide sequences can be optimized for expression in any microorganism.

Methodologies for the construction of plant expression cassettes as well as the introduction of foreign DNA into plants are well known in the art. Such expression cassettes may include one or more regulatory sequences such as promoters, terminators, enhancers, leader sequences, introns, plant translational consensus sequences and other regulatory sequences operably linked to the coding sequence encoding the pesticidal protein as described herein. It is further recognized that promoters or terminators of the Vip gene can be used in expression cassettes.

Generally, for the introduction of foreign DNA into plants Ti plasmid vectors have been utilized for the delivery of foreign DNA as well as direct DNA uptake, liposomes, electroporation, micro-injection, and the use of microprojectiles. Such methods had been published in the art. It is understood that the method of transformation will depend upon the plant cell to be transformed.

### Regulatory Sequences

It is further recognized that the components of the expression cassette may be modified to increase expression. For example, truncated sequences, nucleotide substitutions or other modifications maybe employed. Transcriptional and translational regulatory signals include, but are not limited to, promoters, transcriptional initiation start sites, operators, activators, enhancers, other regulatory elements, ribosomal binding sites, an initiation codon, termination signals, and the like.

It may be beneficial to include 5′ leader sequences in the expression cassette construct. Such leader sequences can act to enhance translation. Translational leaders are known in the art and include but not limited to Picomavirus leaders, Polyvirus leaders, for example, TEV leader (Tobacco Etch Virus); MDMV leader (Maize Dwarf Mosaic Virus), untranslated leader from the coat protein mRNA of alfalfa mosaic virus (AMV RNA 4), Tobacco mosaic virus leader (TMV), and Maize Chlorotic Mottle Virus leader (MCMV).

The polynucleotide/DNA construct will include in the 5′ to 3′ direction of transcription: a transcriptional and translational initiation region (i.e., a promoter), a DNA sequence of the embodiments, and a transcriptional and translational termination region (i.e., termination region) functional in the organism serving as a host. The transcriptional initiation region (i.e., the promoter) may be native, analogous, foreign or heterologous to the host organism and/or to the sequence of the embodiments. Additionally, the promoter may be the natural sequence or alternatively a synthetic sequence. The term “foreign” as used herein indicates that the promoter is not found in the native organism into which the promoter is introduced. Where the promoter is “foreign” or “heterologous” to the sequence of the embodiments, it is intended that the promoter is not the native or naturally occurring promoter for the operably linked sequence of the embodiments.

A number of promoters can be used in the practice of the embodiments. The promoters can be selected based on the desired outcome. The nucleotide sequence of the invention can be combined with constitutive, tissue-preferred, inducible, or other promoters for expression in the host organism. Suitable constitutive promoters for use in a plant host cell include, for example, the core CaMV 35S promoter; rice actin; ubiquitin; ALS promoter etc.

Depending on the desired outcome, it may be beneficial to express the gene from an inducible promoter. Of particular interest for regulating the expression of the nucleotide sequences of the embodiments in plants are wound-inducible promoters. Such wound-inducible promoters, may respond to damage caused by insect feeding, and include potato proteinase inhibitor (pin II) gene; wun1 and wun2, win1 and win2; WIP1; MPI gene etc.

Additionally, pathogen-inducible promoters may be employed in the methods and nucleotide constructs of the embodiments. Such pathogen-inducible promoters include those from pathogenesis-related proteins (PR proteins), which are induced following infection by a pathogen; e.g., PR proteins, SAR proteins, β-1,3-glucanase, chitinase, etc.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters.

A promoter that has “preferred” expression in a particular tissue is expressed in that tissue to a greater degree than in at least one other plant tissue. Some tissue-preferred promoters show expression almost exclusively in the particular tissue. Tissue-preferred promoters can be utilized to target enhanced pesticidal protein expression within a particular plant tissue. Such promoters can be modified, if necessary, for weak expression.

Example of some of the tissue specific promoters includes but is not limited to leaf-preferred promoters, root specific or root preferred promoters, seed specific or seed preferred promoters, pollen specific promoters, and pith specific promoters.

Root-preferred or root-specific promoters are known and can be selected from the available from the literature or isolated de novo from various compatible species.

“Seed-preferred” promoters include both “seed-specific” promoters (those promoters active during seed development such as promoters of seed storage proteins) as well as “seed-germinating” promoters (those promoters active during seed germination). Gamma-zein and Glob-1 are endosperm-specific promoters. For dicots, seed-specific promoters include, but are not limited to β-phaseolin, β.—conglycinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc.

Where low level expression is desired, weak promoters will be used. Generally, the term “weak promoter” as used herein refers to a promoter that drives expression of a coding sequence at a low level. Such weak constitutive promoters include, for example the core promoter of the Rsyn7 promoter, the core 35S CaMV promoter etc.

Termination regions are available from the Ti-plasmid of A. tumefaciens, such as the octopine synthase (OCS) and nopaline synthase (NOS) termination regions.

The DNA expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region); potyvirus leaders, for example, TEV leader (Tobacco Etch Virus), MDMV leader (Maize Dwarf Mosaic Virus), untranslated leader from the coat protein mRNA of alfalfa mosaic virus (AMV RNA 4); tobacco mosaic virus leader (TMV); and maize chlorotic mottle virus leader (MCMV).

In one specific embodiment of the invention disclosed and claimed herein, the tissue-preferred or tissue-specific promoter is operably linked to a synthetic DNA sequence of the disclosure encoding the insecticidal protein, and a transgenic plant stably transformed with at least one such recombinant molecule. The resultant plant will be resistant to particular insects which feed on those parts of the plant in which the DNA(s) is (are) expressed.

It is recognized that the genes encoding the pesticidal proteins can be used to transform insect pathogenic organisms. Such organisms include Baculoviruses, fungi, protozoa, bacteria and nematodes.

A number of ways are available for introducing a gene expressing the pesticidal protein into the microorganism host under conditions which allow for stable maintenance and expression of the gene. For example, expression cassettes can be constructed which include the DNA constructs of interest operably linked with the transcriptional and translational regulatory signals for expression of the DNA constructs, and a DNA sequence homologous with a sequence in the host organism, whereby integration will occur, and/or a replication system which is functional in the host, whereby integration or stable maintenance will occur.

### Selectable Marker Gene

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (nptll) and hygromycin phosphotransferase (hptll), as well as genes conferring resistance to herbicidal compounds, such as glufosinate ammonium, bromoxynil, imidazolinones, and 2,4-dichlorophenoxyacetate (2,4-D). Additional examples of suitable selectable marker genes include, but are not limited to, genes encoding resistance to chloramphenicol, methotrexate, streptomycin, spectinomycin, bleomycin, sulphonamide, bromoxynil, glyphosate, phosphinothricin.

The above list of selectable marker genes is not meant to be limiting. Any selectable marker gene can be used in the embodiments.

### DNA Constructs and Vectors

The DNA/nucleotide sequences of the inventions are provided in DNA constructs for expression in the organism of interest. The construct will include 5′ and 3′ regulatory sequences operably linked to a sequence of the invention.

Such a polynucleotide construct is provided with a plurality of restriction sites for insertion of the DNA sequences encoding the Vip toxin protein to be under the transcriptional regulation of the regulatory regions. The polynucleotide construct may additionally contain selectable marker genes. The construct may additionally contain at least one additional gene to be co-transformed into the desired organism. Alternatively, the additional gene(s) can be provided on multiple polynucleotide constructs.

In preparing the DNA construct/expression cassette, the various DNA fragments may be manipulated so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

According to the present invention, the DNA construct/expression cassette disclosed herein may be inserted to the recombinant expression vector. The expression “recombinant expression vector” means a bacteria plasmid, a phage, a yeast plasmid, a plant cell virus, a mammalian cell virus, or other vector. In general, as long as it can be replicated and stabilized in a host, any plasmid or vector can be used. Important characteristic of the expression vector is that it has a replication origin, a promoter, a marker gene, and a translation control element.

A large number of cloning vectors comprising a replication system in E. coli and a marker that permits selection of the transformed cells are available for preparation for the insertion of foreign genes into higher plants. The vectors comprise, for example, pBR322, pUC series, M13mp series, pACYC184, inter alia. Accordingly, the DNA fragment having the sequence encoding the Bt toxin protein can be inserted into the vector at a suitable restriction site. The resulting plasmid is used for transformation into E. coli. The E. coli cells are cultivated in a suitable nutrient medium, then harvested and lysed. The plasmid is recovered. Sequence analysis, restriction analysis, electrophoresis, and other biochemical-molecular biological methods are generally carried out as methods of analysis. After each manipulation, the DNA sequence used can be cleaved and joined to the next DNA sequence. Each plasmid sequence can be cloned in the same or other plasmids. Depending on the method of inserting desired genes into the plant, other DNA sequences may be necessary. If, for example, the Ti or Ri plasmid is used for the transformation of the plant cell, then at least the right border, but often the right and the left border of the Ti or Ri plasmid T-DNA, has to be joined as the flanking region of the genes to be inserted.

The expression vector comprising the nucleotide sequence of the disclosure and a suitable signal for regulating transcription/translation can be constructed by a method which is well known to a person in the art. Examples of such method include an in vitro recombination DNA technique, a DNA synthesis technique, and an in vivo recombination technique. The DNA sequence can be effectively linked to a suitable promoter in the expression vector in order to induce synthesis of mRNA. Furthermore, the expression vector may contain, as a site for translation initiation, a ribosome binding site and a transcription terminator.

A preferred example of the recombinant vector of the present invention is Ti-plasmid vector which can transfer a part of itself, i.e., so called T-region, to a plant cell when the vector is present in an appropriate host such as Agrobacterium tumefaciens. Other types of Ti-plasmid vector are currently used for transferring a hybrid gene to protoplasts that can produce a new plant by appropriately inserting a plant cell or hybrid DNA to a genome of a plant.

Expression vector may comprise at least one selectable marker gene. The selectable marker gene is a nucleotide sequence having a property based on that it can be selected by a common chemical method. Every gene which can be used for the differentiation of transformed cells from non-transformed cell can be a selective marker. Example includes, a gene resistant to herbicide such as glyphosate and Phosphinothricin, and a gene resistant to antibiotics such as kanamycin, hygromycin, G418, bleomycin, and chloramphenicol, but not limited thereto.

For the recombinant vector of the present invention, a promoter can be any of CaMV 35S, actin, or ubiquitin promoter, but not limited thereto. Since a transformant can be selected with various mechanisms at various stages, a constitutive promoter can be preferable for the present invention. Therefore, a possibility for choosing a constitutive promoter is not limited herein.

For the recombinant vector of the present invention, any conventional terminator can be used. Examples thereof include nopaline synthase (NOS), rice α-amylase RAmy1 A terminator, phaseoline terminator, and a terminator for octopine gene of Agrobacterium tumefaciens, etc., but are not limited thereto. Regarding the necessity of terminator, it is generally known that such region can increase reliability and an efficiency of transcription in plant cells. Therefore, the use of terminator is highly preferable in view of the contexts of the present invention.

One skilled in the art will know that the DNA construct and vector disclosed herein can be used for production of insect resistant transgenic plants and/or production of insecticidal composition, wherein the composition comprises may comprise Bacillus thuringiensis cells comprising the said nucleotide sequence or any other microorganism capable of expressing the nucleotide sequence disclosed herein to produce the Vip insecticidal protein disclosed herein.

### Host Cell and Recombinant Cell

Suitable host cells may include prokaryotes and/or eukaryotes, normally being limited to those cells which do not produce substances toxic to higher organisms, such as mammals. However, organisms which produce substances toxic to higher organisms could be used, where the toxin is unstable or the level of application sufficiently low as to avoid any possibility of toxicity to a mammalian host. As hosts, of particular interest will be the prokaryotes and the lower eukaryotes, such as fungi. Examples of prokaryotic cells, both Gram-negative and -positive, include Enterobacteriaceae, such as Escherichia, Erwinia, Shigella, Salmonella, and Proteus; Bacillaceae; Rhizobiceae, such as Rhizobium; Spirillaceae, such as photobacterium, Zymomonas, Serratia, Aeromonas, Vibrio, Desulfovibrio, Spirillum; Lactobacillaceae; Pseudomonadaceae, such as Pseudomonas and Acetobacter; Azotobacteraceae and Nitrobacteraceae. Among eukaryotes are fungi, such as Phycomycetes and Ascomycetes, which includes yeast, such a Saccharomyces and Schizosaccharromyces; and Basidiomycetes yeast, such as Rhodotorula, Aureobasidium, Sporobolomyces, and the like.

Host organisms of particular interest include yeast, such as Rhodotorula sp., Aureobasidium sp., Saccharomyces sp., and Sporobolomyces sp.; phylloplane organisms such as Pseudomonas sp., Erwinia sp. and Flavobacterium sp.; or such other organisms as Escherichia, LactoBacillus sp., Bacillus sp., and the like. Specific organisms include Pseudomonas aeurginosa, Pseudomonas fluorescens, Saccharomyces cerevisiae, Bacillus thuringiensis, Escherichia coli, Bacillus subtilis, and the like.

The nucleotide sequence encoding the Vip protein disclosed herein can be introduced into micro-organisms that multiply on plants (epiphytes) to deliver VIP proteins to potential target pests. Epiphytes can be gram-positive or gram-negative bacteria.

Various expression systems can be designed so that the Vip protein disclosed herein are secreted outside the cytoplasm of gram negative bacteria, E. coli, for example. Secretion of the said protein outside the cytoplasm avoids potential toxic effects of the Vip protein expressed within the cytoplasm, it can increase the level of the Vip protein expressed and it can aid in efficient purification of the Vip protein.

The Vip protein disclosed herein can be made to be secreted in E. coli, for example, by fusing an appropriate E. coli signal peptide to the amino-terminal end of the Vip protein signal peptide or replacing the Vip signal peptide with the E. coli signal peptide. Signal peptides recognized by E. coli can be found in proteins already known to be secreted in E. coli.

The embodiments further encompass a microorganism that is transformed with the nucleotide sequence encoding the insecticidal Vip protein of the invention, with an expression cassette comprising the nucleotide sequence, or with a vector comprising the expression cassette. In some embodiments, the microorganism is one that multiplies on plants. An embodiment of the invention relates to an encapsulated pesticidal protein which comprises a transformed microorganism capable of expressing the insecticidal Vip protein of the invention.

A further embodiment relates to a transformed organism such as an organism selected from the group consisting of plant and insect cells, bacteria, yeast, baculoviruses, protozoa, nematodes, and algae. The transformed organism comprises the DNA sequence of the invention, an expression cassette comprising the said DNA sequence, or a vector comprising the said expression cassette, which may be stably incorporated into the genome of the transformed organism.

It is recognized that the genes encoding the insecticidal Vip protein of the invention can be used to transform insect pathogenic organisms. Such organisms include baculoviruses, fungi, protozoa, bacteria, and nematodes.

The nucleotide sequence(s) encoding the insecticidal Vip protein of the invention may be introduced via a suitable vector into a microbial host, and said host applied to the environment, or to plants or animals. The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid, or mitochondrial DNA), converted into an autonomous replicon, or transiently expressed (e.g., transfected mRNA).

A number of ways are available for introducing a foreign DNA expressing the pesticidal protein into the microorganism host under conditions that allow for stable maintenance and expression of the DNA. For example, expression cassettes can be constructed which include the nucleotide constructs of interest operably linked with the transcriptional and translational regulatory signals for expression of the nucleotide constructs, and a nucleotide sequence homologous with a sequence in the host organism, whereby integration will occur, and/or a replication system that is functional in the host, whereby integration or stable maintenance will occur.

### Insect Pests

For purposes of the present invention, pests include but are not limited to insects, fungi, bacteria, nematodes, mites, ticks, protozoan pathogens, animal-parasitic liver flukes, and the like. Insect pests include insects selected from the orders Lepidoptera, Coleoptera, Diptera, Homoptera, Hymenoptera, Mallophaga, Hemiptera, Orthoptera, Thysanoptera, Dermaptera, Isoptera, Anoplura, Siphonaptera, Trichoptera, etc., particularly Coleoptera and Lepidoptera.

Proteins that possess insecticidal activity against a wide spectrum of lepidopteran pests, including 805 but not limited to black cutworm (BCW, Agrotis ipsilon), fall armyworm (FAW, Spodoptera frugiperda), tobacco budworm (TBW, Heliothis virescens), sugarcane borer (SCB, Diatraea saccharalis), lesser cornstalk borer (LCB, Elasmopalpus lignosellus), and corn earworm (CEW, Helicoverpa zea).

Lepidoptera (moths and butterflies) are the second most diverse pest insect order outnumbered only by the beetles. There is hardly any cultivated plant that is not attacked by at least one lepidopteran pest. As pollinators of many plants, adult moths and butterflies are usually beneficial insects that feed on nectar using their siphoning proboscis. The caterpillars however almost always have chewing mouthparts that are suitable for feeding on various parts of a plant. Most caterpillars are defoliators or miners of succulent plant tissues.

Examples of lepidopteran pest includes but not limited to Agrotis ipsilon, Agrotis orthogonia, Agrotis segetum, Anticarsia gemmatalis, Artogeia rapae, Ostrinia nubilalis, Ostrinia furnacalis, Helicoverpa zea, Helicoverpa armigera, Heliothis virescens, Spodoptera frugiperda, Spodoptera exigua, Spodoptera ornithogalli, Spodoptera praefica, Spodoptera eridania, Diatraea grandiosella, Diatraea saccharalis, Elasmopalpus lignosellus, Sesamia nonagroides, Chilo partellus, Chilo suppressalis, Feltia subterranean, Pseudaletia unipunctata, Suleima helianthana, Homoeosoma electellum, Pectinophora gossypiella, Scirpophaga sp., Pseudoplusia includens, Plathypena scabra, Peridroma saucia, Papaipema nebris, Phthorimaea operculella, Pieris brassicae, Plutella xylostella, Platynota idaeusalis, Trichoplusia ni, Keiferia lycopersicella, Manduca sexta, Manduca quinquemaculata, Mamestra configura, Endopiza viteana, and Cydia pomonella.

### Coleoptera (Beetles)

Examples of Coleopteran pest includes but not limited to Diabrotica virgifera virgifera, Diabrotica longicornis barberi, Diabrotica undecimpunctata howardi, Melanotus spp., Cyclocephala borealis, Cyclocephala immaculata, Popillia japonica, Chaetocnema pulicaria, Sphenophorus maidis, Phyllophaga crinita, Eleodes spp., Conoderus spp., Aeolus spp., Oulema melanopus, Chaetocnema pulicaria, Sphenophorus maidis, Oulema melanopus, Hypera punctata, Diabrotica undecimpunctata howardi, Zygogramma exciamationis, Bothyrus gibbosus, Anthonomus grandis, Colaspis brunnea, colaspis Lissorhoptrus oryzopyhilus, Sitophilus oryzae, Oulema oryzae, Epilachna varivestis, Leptinotarsa decemlineata, Epitrix hirtipennis, Phyllotreta cruciferae, Phyllotreta pusilla, Anthonomus eugenii, Leptinotarsa decemlineata, Epitrix cucumeris, Hemicrepidus memnonius, Melanpotus spp., and Ceutorhychus assimilis, Homoptera (Whiteflies, Aphids) Examples of Homopteran pest includes but not limited to Rhopalosiphum maidis, Anuraphis maidiradicis, Sipha flava, Schizaphis graminum, Macrosiphum avenae, Macrosiphum euphorbiae, Aphis gossypii, Pseudatomoscelis seriatus, Trialeurodes abutilonea, Nephotettix nigropictus, Nilaparvata lugens, Sogatella furcifera, Laodelphaax striatellus, Myzus persicae, Empoasca fabae, Schizaphis graminum, Brevicoryne brassicae, Trileurodes vaporariorum, Bemisia tabaci, Bemisia argentifolii, Paratrioza cockerelli, Cavariella aegopodii, Brevicoryne brassicae, Pemphigus popullivenae, Dysaphis plantaginea, and Saccharosydne saccharivora.

### Bioassay

A wide variety of bioassay techniques are known to one skilled in the art. General procedures include addition of the experimental compound or organism to the diet source in an enclosed container. Pesticidal activity can be measured by, but is not limited to, changes in mortality, weight loss, attraction, repellency and other behavioural and physical changes after feeding and exposure for an appropriate length of time. Bioassays described herein can be used with any feeding insect pest in the larval or adult stage.

### Insecticidal Composition

Compositions of the embodiments find use in protecting plants, seeds, and plant products in a variety of ways. For example, the compositions can be used in a method that involves placing an effective amount of the pesticidal composition in the environment of the pest by a procedure selected from the group consisting of spraying, dusting, broadcasting, or seed coating.

Before plant propagation material (fruit, tuber, bulb, corm, grains, seed), but especially seed, is sold as a commercial product, it is customarily treated with a protectant coating comprising herbicides, insecticides, fungicides, bactericides, nematicides, molluscicides, or mixtures of several of these preparations, if desired together with further carriers, surfactants, or application-promoting adjuvants customarily employed in the art of formulation to provide protection against damage caused by bacterial, fungal, or animal pests. In order to treat the seed, the protectant coating may be applied to the seeds either by impregnating the tubers or grains with a liquid formulation or by coating them with a combined wet or dry formulation. In addition, in special cases, other methods of application to plants are possible, e.g., treatment directed at the buds or the fruit.

The plant seed of the embodiments comprising a nucleotide sequence encoding the pesticidal protein of the embodiments may be treated with a seed protectant coating comprising a seed treatment compound, such as, for example, captan, carboxin, thiram, methalaxyl, pirimiphos-methyl, and others that are commonly used in seed treatment. In one embodiment, a seed protectant coating comprising a pesticidal composition of the embodiments is used alone or in combination with one of the seed protectant coatings customarily used in seed treatment.

The compositions of the embodiments can be in a suitable form for direct application or as a concentrate of primary composition that requires dilution with a suitable quantity of water or other diluent before application. The pesticidal concentration will vary depending upon the nature of the particular formulation, specifically, whether it is a concentrate or to be used directly. The composition contains 1 to 98% of a solid or liquid inert carrier, and 0 to 50% or 0.1 to 50% of a surfactant. These compositions may be administered at the labelled rate for the commercial product.

In the embodiments, a transformed microorganism (which includes whole organisms, cells, spore(s) such as Bacillus thuringiensis transformed with the nucleotide sequences disclosed herein, pesticidal protein(s), pesticidal component(s), pest-affecting component(s), mutant(s), living or dead cells and cell components, including mixtures of living and dead cells and cell components, and including broken cells and cell components) or an isolated pesticidal protein can be formulated with an acceptable carrier into a pesticidal composition(s) that is, for example, a suspension, a solution, an emulsion, a dusting powder, a dispersible granule or pellet, a wettable powder, and an emulsifiable concentrate, an aerosol or spray, an impregnated granule, an adjuvant, a coatable paste, a colloid, and also encapsulations in, for example, polymer substances. Such formulated compositions may be prepared by such conventional means as desiccation, lyophilization, homogenization, extraction, filtration, centrifugation, sedimentation, or concentration of a culture of cells comprising the polypeptide.

Such compositions disclosed above may be obtained by the addition of a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors, or other preparations that influence plant growth. One or more agrochemicals including, but not limited to, herbicides, insecticides, fungicides, bactericides, nematicides, molluscicides, acaricides, plant growth regulators, harvest aids, and fertilizers, can be combined with carriers, surfactants or adjuvants customarily employed in the art of formulation or other components to facilitate product handling and application for particular target pests. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients of the embodiments are normally applied in the form of compositions and can be applied to the crop area, plant, or seed to be treated. For example, the compositions of the embodiments may be applied to grain in preparation for or during storage in a grain bin or silo, etc. The compositions of the embodiments may be applied simultaneously or in succession with other compounds. Methods of applying an active ingredient of the embodiments or an agrochemical composition of the embodiments that contains at least one of the pesticidal proteins produced by the bacterial strains of the embodiments include, but are not limited to, foliar application, seed coating, and soil application. The number of applications and the rate of application depend on the intensity of infestation by the corresponding pest.

In a further embodiment, the compositions, as well as the transformed microorganisms and pesticidal protein of the embodiments, can be treated prior to formulation to prolong the pesticidal activity when applied to the environment of a target pest as long as the pre-treatment is not deleterious to the pesticidal activity. Such treatment can be by chemical and/or physical means as long as the treatment does not deleteriously affect the properties of the composition(s).

Examples of chemical reagents include but are not limited to halogenating agents; aldehydes such as formaldehyde and glutaraldehyde; anti-infectives, such as zephiran chloride; alcohols, such as isopropanol and ethanol.

In other embodiments, it may be advantageous to treat the toxin polypeptides with a protease, for example trypsin, to activate the protein prior to application of a pesticidal protein composition of the embodiments to the environment of the target pest. Methods for the activation of protoxin by a serine protease are well known in the art.

The compositions (including the transformed microorganisms and pesticidal protein of the embodiments) can be applied to the environment of an insect pest by, for example, spraying, atomizing, dusting, scattering, coating or pouring, introducing into or on the soil, introducing into irrigation water, by seed treatment or general application or dusting at the time when the pest has begun to appear or before the appearance of pests as a protective measure. For example, the pesticidal protein and/or transformed microorganisms of the embodiments may be mixed with grain to protect the grain during storage. It is generally important to obtain good control of pests in the early stages of plant growth, as this is the time when the plant can be most severely damaged. The compositions of the embodiments can conveniently contain another insecticide if this is thought necessary. In one embodiment, the composition is applied directly to the soil, at a time of planting, in granular form of a composition of a carrier and dead cells of a Bacillus strain or transformed microorganism of the embodiments. Another embodiment is a granular form of a composition comprising an agrochemical such as, for example, an herbicide, an insecticide, a fertilizer, an inert carrier, and dead cells of a Bacillus strain or transformed microorganism of the embodiments.

Those skilled in the art will recognize that not all compounds are equally effective against all pests. Compounds of the embodiments display activity against insect pests, which may include economically important agronomic, forest, greenhouse, nursery, ornamentals, food and fibre, public and animal health, domestic and commercial structure, household, and stored product pests. Insect pests include insects from the order Lepidoptera. Larvae of the order Lepidoptera include, but are not limited to, armyworms, cutworms, loopers, and heliothines in the family Noctuidae Spodoptera frugiperda J E Smith (fall armyworm); S. exigua Hubner (beet armyworm); S. litura Fabricius (tobacco cutworm, cluster caterpillar); Mamestra configurata Walker (bertha armyworm); M. brassicae Linnaeus(cabbage moth); Agrotis ipsilon Hufnagel (black cutworm); A. orthogonia Morrison (western cutworm); A. subterranea Fabricius (granulate cutworm); Alabama argillacea Hubner (cotton leaf worm); Trichoplusia ni Hubner (cabbage looper); Pseudoplusia includens Walker (soybean looper); Anticarsia gemmatalis Hubner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messoria Harris (darksided cutworm); Earias insulana Boisduval (spiny bollworm); E. vittella Fabricius (spotted bollworm); Helicoverpa armigera Hubner (American bollworm); H. zea Boddie (corn earworm or cotton bollworm); Melanchra picta Harris (zebra caterpillar); Egira (Xylomyges) curialis Grote (citrus cutworm); borers, casebearers, webworms, coneworms, and skeletonizers from the family Pyralidae Ostrinia nubilalis Hubner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); C. partellus,(sorghum borer); Corcyra cephalonica Stainton (rice moth); Crambus caliginosellus Clemens (corn root webworm); C. teterrellus Zincken (bluegrass webworm); Cnaphalocrocis medinalis Guenee (rice leaf roller); Desmia funeralis Hubner (grape leaffolder); Diaphania hyalinata Linnaeus (melon worm); D. nitidalis Stoll (pickleworm); Diatraea grandiosella Dyar (southwestern corn borer), D. saccharalis Fabricius (surgarcane borer); Eoreuma loftini Dyar (Mexican rice borer); Ephestia elutella Hubner (tobacco (cacao) moth); Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia grisella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hubner (Indian meal moth); Scirpophaga incertulas Walker (yellow stem borer); Udea rubigalis Guenee (celery leaftier); and leafrollers, budworms, seed worms, and fruit worms in the family Tortricidae Acleris gloverana Walsingham (Western blackheaded budworm); A. variana Fernald (Eastern blackheaded budworm); Archips argyrospila Walker (fruit tree leaf roller); A. rosana Linnaeus (European leaf roller); and other Archips species, Adoxophyes orana Fischer von Rosslerstamm (summer fruit tortrix moth); Cochylis hospes Walsingham (banded sunflower moth); Cydia latiferreana Walsingham (filbertworm); C. pomonella Linnaeus (codling moth); Platynota flavedana Clemens (variegated leafroller); P. stultana Walsingham (omnivorous leafroller); Lobesia botrana Denis & Schiffermuller (European grape vine moth); Spilonota ocellana Denis & Schiffermuller (eyespotted bud moth); Endopiza viteana Clemens (grape berry moth); Eupoecilia ambiguella Hubner (vine moth); Bonagota salubricola Meyrick (Brazilian apple leafroller); Grapholita molesta Busck (oriental fruit moth); Suleima helianthana Riley (sunflower bud moth); Argyrotaenia spp.; Choristoneura spp.

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guerin-Meneville (Chinese Oak Silkmoth); Bombyx mori Linnaeus (Silkworm); Bucculatrix thurberiella Busck (cotton leaf perforator); Collas eurytheme Boisduval (alfalfa caterpillar); Datana integerrima Grote & Robinson (walnut caterpillar); Dendrolimus sibiricus Tschetwerikov (Siberian silk moth), Ennomos subsignaria Hubner (elm spanworm); Erannis tiliaria Harris (linden looper); Euproctis chtysorrhoea Linnaeus (browntail moth); Harrisina americana Guerin-Meneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Manduca quinquemaculata Haworth (five spotted hawk moth, tomato hornworm); M. sexta Haworth (tomato hornworm, tobacco hornworm); Operophtera brumata Linnaeus (winter moth); Paleacrita vemata Peck (spring cankerworm); Papilio cresphontes Cramer (giant swallowtail, orange dog); Phryganidia californica Packard (California oakworm); Phyllocnistis citrella Stainton (citrus leafminer); Phyllonotycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval & Leconte (Southern cabbageworm); Sabulodes aegrotata Guenee (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absolute Meyrick (tomato leafminer); Yponomeuta padella Linnaeus (ermine moth); Heliothis subflexa Guenee; Malacosoma spp. and Orgyia spp.

Insect pests may be tested for pesticidal activity of compositions of the embodiments in early developmental stages, e.g., as larvae or other immature forms. The insects may be reared in total darkness at from about 20° C. to about 30° C. and from about 50% to about 70% relative humidity.

Methods of rearing insect larvae and performing bioassays are well known to one of ordinary skill in the art.

A method for controlling insects, particularly Lepidoptera, in accordance with this invention can comprise applying (e.g., spraying) the insecticidal/pesticidal composition disclosed herein to an area or plant to be protected to a locus (area) to be protected, comprising host cells transformed with the nucleotide sequences of the invention. The locus to be protected can include, for example, the habitat of the insect pests or growing vegetation or an area where vegetation is to be grown.

The present disclosure further relates to a method for controlling lepidopteran corn insect pests, which method comprises applying the insecticidal/pesticidal composition disclosed herein to an area or plant to be protected to an area or plant to be protected, by planting a corn plant transformed with the nucleotide sequence(s) of the invention, or spraying a composition containing the Vip protein of the invention. The invention also relates to use of the composition disclosed herein, against Lepidopteran corn insect pests to minimize damage to corn plants.

The present disclosure relates to a method for controlling lepidopteran eggplant insect pests, which method comprises applying the insecticidal/pesticidal composition disclosed herein to an area or plant to be protected, by planting eggplant plants transformed with the nucleotide sequence(s) of the invention, or spraying a composition containing a the protein of the invention.

The invention also relates to use of the composition of the invention against Lepidopteran eggplant insect pests to minimize damage to eggplant plants.

The present disclosure also relates to a method for controlling lepidopteran rice insect pests, particularly Lepidopteran rice stem borers, rice leaffolders rice skippers, rice cutworms, rice armyworms, or rice caseworms, preferably an insect selected from the group consisting of: Chilo suppressalis, Chilo partellus, Scirpophaga incertulas, Sesamia inferens, Cnaphalocrocis medinalis, Marasmia patnalis, Marasmia exigua, Marasmia ruralis, Scirpophaga innotata, which method comprises applying the insecticidal/pesticidal composition disclosed herein to an area or plant to be protected to an area or plant to be protected, by planting a rice plant transformed with the nucleotide sequence(s) of the invention, or spraying a composition containing the Vip protein of the invention. The invention also relates to use of the composition disclosed herein, against Lepidopteran rice insect pests to minimize damage to rice plants.

The present disclosure further relates to a method for controlling lepidopteran tomato insect pests, particularly lepidopteran Helicoverpa armigera which method comprises applying the insecticidal/pesticidal composition disclosed herein to an area or plant to be protected to an area or plant to be protected, by planting a tomato plant transformed with the nucleotide sequence(s) of the invention, or spraying a composition containing the Vip protein of the invention. The invention also relates to use of the composition disclosed herein, against lepidopteran tomato insect pests to minimize damage to tomato plants.

The present disclosure further relates to a method for controlling lepidopteran cotton insect pests, which method comprises applying the insecticidal/pesticidal composition disclosed herein to an area or plant to be protected to an area or plant to be protected, by planting a cotton plant transformed with the nucleotide sequence(s) of the invention, or spraying a composition containing the Vip protein of the invention. The invention also relates to use of the composition disclosed herein, against Lepidopteran cotton insect pests to minimize damage to cotton plants.

To obtain the insecticidal protein (SEQ ID NO:1, SEQ ID NO:7 and SEQ ID NO:11), cells of the recombinant hosts expressing the Vip proteins can be grown in a conventional manner on a suitable culture medium. The toxin can then be separated and purified by standard techniques such as chromatography, extraction, electrophoresis, or the like.

Accordingly, the present invention provides compositions and methods for affecting insect pests, particularly plant pests. More specifically, the invention provides the polynucleotides that encodes the Vip protein as disclosed herein against insect pests such as, but not limited to, insect pests of the order Lepidopteran pests, such as Spodoptera frugiperda (fall armyworm), Helicoverpa armigera—the cotton bollworm and corn earworm, Cnaphalocrocis medinalis—the rice leaffolder, and Scirpophaga incertulas—the rice yellow stem borer, and Pectinophora gossypiella.

In accordance with the present invention, a nucleotide sequences encoding the Vip proteins derived from Bacillus thuringiensis has been identified. One of the preferred embodiments of the present provides an insecticidal protein having an amino acid sequence selected from the group consisting of SEQ ID NO: 1, SEQ ID NO: 7 and SEQ ID NO: 11, when expressed in a plant, results in an insecticidal activity.

In another embodiment there is provided a recombinant nucleic acid molecule comprising a nucleotide sequence encoding the protein having an amino acid sequence selected from the group consisting of SEQ ID NO: 1, SEQ ID NO: 7 and SEQ ID NO: 11. In another embodiment there is provided a recombinant nucleic acid molecule comprising a nucleotide sequence encoding the proteins as disclosed herein, wherein said nucleic acid molecule comprises the nucleotide sequence selected from the group consisting of SEQ ID NO: 2, SEQ ID NO: 5, SEQ ID NO: 6, SEQ ID NO: 8, SEQ ID NO: 9, SEQ ID NO: 10, SEQ ID NO: 12, SEQ ID NO: 13 and SEQ ID NO: 14. In yet another embodiment there is provided a recombinant nucleic acid molecule comprising a nucleotide sequence encoding the proteins as disclosed herein, wherein said nucleic acid molecule comprises the nucleotide sequence selected from the group consisting of SEQ ID NO: 5, SEQ ID NO: 6, SEQ ID NO: 9, SEQ ID NO: 10, SEQ ID NO: 13 and SEQ ID NO: 14.

In further embodiment there is provided the recombinant nucleic acid molecule as disclosed herein, wherein the nucleotide sequence is a synthetic sequence which has been designed for optimum expression in a plant.

Yet another embodiment of the present invention relates to the nucleotide sequence which encodes a protein disclosed in the present invention, wherein said sequence has a complement which hybridizes to a coding sequence selected from the group consisting of SEQ ID NO: 2, SEQ ID NO: 5, SEQ ID NO: 6, SEQ ID NO: 8, SEQ ID NO: 9, SEQ ID NO: 10, SEQ ID NO: 12, SEQ ID NO: 13 and SEQ ID NO: 14 under hybridization conditions of 68° C. followed by washing at 68° C. in 2×SSC containing 0.1% SDS.

Yet another embodiment of the present invention relates to the nucleotide sequence which encodes a protein disclosed in the present invention, wherein said sequence has a complement which hybridizes to a coding sequence selected from the group consisting of SEQ ID NO: 2, SEQ ID NO: 5, and SEQ ID NO: 6 under hybridization conditions of 37° C. to 68° C. followed by washing at 37° C. to 68° C. in 2×SSC containing 0.1% SDS.

Yet another embodiment of the present invention relates to the nucleotide sequence which encodes a protein disclosed in the present invention, wherein said sequence has a complement which hybridizes to a coding sequence selected from the group consisting of SEQ ID NO: 8, SEQ ID NO: 9, and SEQ ID NO: 10 under hybridization conditions of 37° C. to 68° C. followed by washing at 37° C. to 68° C. in 2×SSC containing 0.1% SDS.

Yet another embodiment of the present invention relates to the nucleotide sequence which encodes a protein disclosed in the present invention, wherein said sequence has a complement which hybridizes to a coding sequence selected from the group consisting of SEQ ID NO: 12, SEQ ID NO: 13 and SEQ ID NO: 14 under hybridization conditions of 37° C. to 68° C. followed by washing at 37° C. to 68° C. in 2×SSC containing 0.1% SDS.

The present invention further provides the nucleotide sequence which encodes a protein disclosed in the present invention, wherein said nucleotide sequence hybridizes under conditions of 0.1×SSPE at 65° C. with the full complement of a nucleotide sequence selected from a group consisting of SEQ ID NO: 2, SEQ ID NO: 5, SEQ ID NO: 6, SEQ ID NO: 8, SEQ ID NO: 9, SEQ ID NO: 10, SEQ ID NO: 12, SEQ ID NO: 13 and SEQ ID NO: 14.

The present invention further provides the nucleotide sequence which encodes a protein disclosed in the present invention, wherein said nucleotide sequence hybridizes under conditions of 0.1×SSPE at 55° C. with the full complement of a nucleotide sequence selected from a group consisting of SEQ ID NO: 2, SEQ ID NO: 5, SEQ ID NO: 6, SEQ ID NO: 8, SEQ ID NO: 9, SEQ ID NO: 10, SEQ ID NO: 12, SEQ ID NO: 13 and SEQ ID NO: 14.

In an another embodiment there is provided the recombinant nucleic acid molecule as disclosed herein, wherein said nucleic acid molecule comprising a promoter operably linked to said nucleotide sequence encoding the insecticidal protein(s) of the present invention.

In an another embodiment there is provided an expression cassette comprising a heterologous promoter operably linked to at least one nucleotide sequence as disclosed in the present invention. Another embodiment of the present invention relates to the expression cassette as disclosed herein, wherein said promoter functions in plants is selected from the group consisting of inducible, constitutive, tissue-preferred and tissue-specific promoters.

Further embodiment of the present invention provides a vector comprising the recombinant nucleic acid molecule as disclosed in the present invention. Further, in another embodiment there is provided a host cell comprising at least one protein as disclosed in the present invention. In further embodiment there is provided a host cell comprising at least one recombinant nucleic acid molecule as disclosed in the present invention. In further embodiment there is provided a host cell comprising or the vector as claimed disclosed in the present invention. The host cell host cell is a bacterium, cynobacterium, virus, fungi, insect, or yeast. Another embodiment of the present invention relates to the host cell as disclosed in the present invention wherein the host cell is Agrobacterium or E. coli.

In an another embodiment of the present invention there is provided a method for producing a plant exhibiting an insecticidal activity, wherein said method comprises at least the step of introducing at least one nucleic acid molecule of the present invention. Another embodiment of the present invention relates to the method for producing a plant exhibiting an insecticidal activity, wherein the method comprises at least the step of introducing at least one nucleic acid molecule of the present invention, wherein the introducing is performed by introgressing into the plant a nucleic acid molecule encoding the protein having the amino acid sequence selected from the group consisting of SEQ ID NO: 1, SEQ ID NO: 7 and SEQ ID NO: 11.

Another embodiment of the present invention relates to a method for producing a plant exhibiting an insecticidal activity, wherein said method comprises at least the step of introducing at least one nucleic acid molecule of the present invention, wherein said introducing is performed by
(a) inserting the recombinant nucleic acid molecule as claimed in claim 2 into a plant cell, wherein said nucleic acid molecule further comprises a promoter functional in a plant cell and a terminator, wherein said promoter and terminator is operably linked to the nucleotide sequence encoding the protein having the amino acid sequence selected from the group consisting of SEQ ID NO: 1, SEQ ID NO: 7 and SEQ ID NO: 11.
(b) obtaining a transformed plant cell from said plant cell of step (a), wherein said transformed plant cell comprises the nucleotide sequence encoding at least one said protein; and
(c) generating a transgenic plant from said transformed plant cell of step (b), wherein said transgenic plant comprises the nucleotide sequence encoding at least one said protein.

In an another embodiment there is provided a transgenic plant comprising a recombinant nucleic acid molecule comprising a nucleotide sequence encoding at least one protein disclosed in the present invention, wherein said plant exhibits an insecticidal activity. In further embodiment of the present invention there is provided a progeny and seed obtained from the transgenic plant as disclosed in the present invention.

The plant is monocot, dicot and/or leguminous plant. The monocot plant includes but not limited to corn, wheat, rice, oat, sorghum, millets, and grass plant. The dicot plant includes but not limited to cotton, brinjal, tomato, canola, soybean, tobacco, a fruit tree, a cruciferous plant, a pepper plant, an ornamental plant, sunflower, cucurbits, pigeon pea, pea, chickpea and melon.

In one of the embodiment of the present invention there is provided a recombinant microorganism comprising a recombinant nucleotide sequence which encodes the insecticidal protein of the present invention. Another embodiment provides a recombinant microorganism comprising a recombinant nucleotide sequence which encodes the insecticidal protein of the present invention, wherein the nucleotide sequence is selected from the group consisting of SEQ ID NO: 2, SEQ ID NO: 5, SEQ ID NO: 6, SEQ ID NO: 8, SEQ ID NO: 9, SEQ ID NO: 10, SEQ ID NO: 12, SEQ ID NO: 13 and SEQ ID NO: 14.

Another embodiment relates to a recombinant microorganism comprising a recombinant nucleotide sequence which encodes the insecticidal protein of the present invention, wherein the nucleotide sequence is selected from a group consisting of SEQ ID NO: 2, SEQ ID NO: 5, SEQ ID NO: 6. Another embodiment relates to a recombinant microorganism comprising a recombinant nucleotide sequence which encodes the insecticidal protein of the present invention, wherein the nucleotide sequence is selected from a group consisting of SEQ ID NO: 8, SEQ ID NO: 9, SEQ ID NO: 10. Another embodiment relates to a recombinant microorganism comprising a recombinant nucleotide sequence which encodes the insecticidal protein of the present invention, wherein the nucleotide sequence is selected from a group consisting of SEQ ID NO: 12, SEQ ID NO: 13, SEQ ID NO: 14.

Further embodiment of the present invention relates to the recombinant microorganism comprising a recombinant nucleotide sequence which encodes the insecticidal protein of the present invention, wherein said microorganism is selected from the group consisting of bacteria, virus, algae and fungi. Further embodiment of the present invention provided the recombinant microorganism comprising a recombinant nucleotide sequence which encodes the insecticidal protein of the present invention and a second nucleotide sequence which encodes a second insecticidal protein. The second insecticidal protein may be delta endotoxin or a vegetative insecticidal protein, and second nucleotide may encode a delta endotoxin protein or a vegetative insecticidal protein. Another embodiment of the present invention provides the said microorganism comprising the second nucleotide sequence encoding a delta endotoxin or a vegetative insecticidal protein.

Further embodiment of the present invention provides a composition comprising the nucleotide sequence which encodes the insecticidal protein of the present invention. Another embodiment of the present invention provides a composition comprising a recombinant nucleotide sequence which encodes the insecticidal protein of the present invention, wherein the nucleotide sequence is selected from the group consisting of SEQ ID NO: 2, SEQ ID NO: 5, SEQ ID NO: 6, SEQ ID NO: 8, SEQ ID NO: 9, SEQ ID NO: 10, SEQ ID NO: 12, SEQ ID NO: 13 and SEQ ID NO: 14.

It is intended that the insecticidal Vip proteins disclosed in the present invention can be used for agricultural purposes, i.e., for protecting plants from insect pest infestation, and more particularly for protecting plants from lepidopteran insect pest infestation.

Another embodiment of the present invention provides the nucleotide sequence which encodes the insecticidal toxin proteins as disclosed herein, wherein said nucleotide sequences have been isolated from Bacillus thuringiensis.

Another embodiment of the present invention provides the nucleotide sequence which encodes the insecticidal toxin proteins as disclosed herein, wherein said nucleotide sequences have been optimized for expression in plants. Another embodiment of the present invention provides a nucleotide sequence which encodes an insecticidal toxin protein as disclosed herein, wherein said nucleotide sequence has been optimized for expression in plants, preferably said nucleotide sequence has been optimized for expression in a monocot plant. Another embodiment of the present invention provides a nucleotide sequence which encodes an insecticidal toxin protein as disclosed herein, wherein said nucleotide sequence has been optimized for expression in plants, preferably said nucleotide sequence has been optimized for expression in a dicot plant. Another embodiment of the present invention provides a nucleotide sequence which encodes an insecticidal toxin protein as disclosed herein, wherein said nucleotide sequence has been optimized for expression in plants, preferably said nucleotide sequence has been optimized for expression in a leguminous plant.

Another embodiment of the present invention provides a nucleotide sequence which encodes an insecticidal protein as disclosed herein, wherein said nucleotide sequence has been optimized for expression in plants, wherein said optimization comprising the steps (i) removing polyadenylation sequences, (ii) adjusting the A and T content of the nucleotide sequence to be from 40% to 49% without modifying the amino acid sequence of the protein, and (iii) modifying codons in the coding sequence to be consistent with the steps (i) and (ii).

Further embodiment of the present invention provides a method for controlling an insect pest comprising contacting said pest with a pesticidal amount of the Vip protein as disclosed herein.

Yet another embodiment of the present invention provides a method of controlling insect infestation in a crop plant and providing insect resistance management, wherein said method comprising contacting said crop plant with an insecticidally effective amount of the composition comprising the Vip protein as disclosed herein.

Another embodiment of the present invention provides a method for expressing in a plant an insecticidal protein toxin as disclosed herein, comprising (a) inserting into the genome of a plant cell a nucleotide sequence comprising in the 5′ to 3′ direction an operably linked recombinant, double-stranded DNA molecule, wherein the recombinant, double-stranded DNA molecule comprises a promoter that functions in the plant cell; a nucleotide sequence encoding the insecticidal amino acid sequence selected from a group consigns of SEQ ID NO: 1; SEQ ID NO: 7 and SEQ ID NO: 11; and a 3′ non-translated nucleotide sequence that functions in the cells of the plant to cause termination of transcription; (b) obtaining a transformed plant cell containing the nucleic acid sequence of step (a); and (c) generating from said transformed plant cell a plant that expresses the insecticidal protein in the transformed plant.

Another embodiment of the present invention provides a seed from the transgenic plant as described herein or a progeny of said seed, wherein said seed and said progeny comprises the nucleic acid sequence that encodes the insecticidal Vip protein having amino acid sequence 1240 selected from a group consigns of SEQ ID NO: 1; SEQ ID NO: 7 and SEQ ID NO: 11.

Another embodiment of the present invention provides a method for expressing in a plant an insecticidal protein as disclosed herein, wherein said plant is a monocot plant including but not limited to corn, wheat, rice, oat, sorghum, millets, and grass plant; a dicot plant including but not limited to cotton, brinjal, tomato, canola, soybean, tobacco, a fruit tree, a cruciferous plant, a pepper plant, an ornamental plant, sunflower, cucurbits, pigeon pea, pea, chickpea and melon.

Yet another embodiment of the present invention provides a biological sample derived from the cell, tissues or seed of the plant obtainable by the method disclosed herein, wherein the sample comprises a detectable amount of the nucleotide sequence of the present invention.

Yet another embodiment of the present invention provides an application of a protein in controlling insect pests, wherein the protein having amino acid sequence is selected from a group consigns of SEQ ID NO: 1; SEQ ID NO: 7 and SEQ ID NO: 11.

Yet another embodiment of the present invention provides use of the nucleotide sequence as disclosed herein for production of insecticidal composition, wherein the composition comprises Bacillus thuringiensis cells comprising the said nucleotide sequences.

Yet another embodiment of the present invention provides use of the nucleotide sequence, the DNA construct and or expression cassette or the plasmid as disclosed herein for production of insect resistant transgenic plants.

Yet another embodiment of the present invention provides use of a composition comprising the insecticidal Vip protein disclosed herein for the control of insect pests.

Yet another embodiment of the present invention provides use of a microorganism comprising insecticidal Vip protein disclosed herein for the control of insect pests, wherein said microorganism is Escherichia coli and/or Bacillus thuringiensis.

Yet another embodiment of the present invention provides use of a microorganism comprising insecticidal Vip protein disclosed herein for the control of insect pests, wherein the microorganism comprises a gene encoding said protein.

Yet another embodiment of the present invention provides use of a microorganism comprising insecticidal Vip protein disclosed herein for the control of insect pests, wherein the microorganism comprises a gene encoding said protein having the amino acid sequence selected from a group consigns of SEQ ID NO: 1; SEQ ID NO: 7 and SEQ ID NO: 11.

These and/or other embodiments of this invention are reflected in the wordings of the claims that form part of the description of the invention.

Various modifications and other embodiments of the present invention can be presented by a person skilled in the art to which these inventions pertain having the benefit of the teachings presented in the descriptions and the associated drawings. Therefore, it is to be understood that the present invention is not to be limited to the specific embodiments disclosed herein and that modifications and other embodiments are intended to be included within the scope of the appended claims.

The following Examples illustrate the invention, and are not provided to limit the invention or the protection sought.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperatures, etc.) but some experimental errors and deviations should be accounted for.

### General Methods

DNA manipulations were done using procedures that are standard in the art. These procedures can often be modified and/or substituted without substantively changing the result. Except where other references are identified, most of these procedures are described in Sambrook et al., Molecular Cloning: A Laboratory Manual, Cold Spring Harbor Laboratory Press, second edition, 1989.

### Example 1: Isolation and Identification of Vip Genes

Degenerate PCR primers (SEQ ID NO: 3 and SEQ ID NO: 4) were designed to identify genes that encode Vegetative Insecticidal Proteins (Vip) protein disclosed herein. The DNA isolated from Bacillus thuringiensis (isolated from soil sample obtained from Telangana, India) was subjected to PCR using these primers. The amplicon obtained after PCR amplification was ligated into pCR2.1-TOPO TA vector (ThermoFisher, USA) and the chemically competent TOP10 E. coli cells were transformed with the recombinant pCR2.1-TOPO plasmid. About 198 colonies were screened by PCR using M13 universal primers. Restriction enzyme digestion analysis was performed to confirm the Vip gene amplified using the degenerate primers. Subsequently sequencing of the amplified product was carried out using the Sanger sequencing method (Eurofins Genomics, India). We could amplify three nucleotide sequences having the nucleotide sequence as set forth in SEQ ID NO: 2, SEQ ID NO: 8 and SEQ ID NO: 12. The amino acid sequence deduced from the nucleotide sequence is as set forth the in SEQ ID NO: 1, SEQ ID NO: 7 and SEQ ID NO: 11 respectively.

For ease of understanding these three Vip proteins were designated as:
Vip Protein-1 (SEQ ID NO: 1); Vip Protein-2 (SEQ ID NO: 7), and Vip Protein-3 (SEQ ID NO: 11)

Forward degenerate primer:
SEQ ID NO: 3
ATGAMYAWGAATAATRCTAARTTAA
Reverse degenerate primer:
SEQ ID NO: 4
YTAHYTAATAGAGAMATCRTAAAA

### Example 2: Cloning and Expression of the Nucleotide Sequences Encoding the Vip Proteins

The nucleotide sequence as set forth in SEQ ID NO: 2 was cloned in pET-32a(+) expression vector to obtain recombinant vector comprising the nucleotide sequence as set forth in SEQ ID NO: 2. Subsequently E. coli cells (BL21(DE3)) were transformed with the recombinant vector to obtain recombinant E. coli cells comprising the recombinant vector comprising of the nucleotide sequence as set forth in SEQ ID NO: 2. The BL21—cells were transformed with 10-50 ng expression vector carrying the nucleotide sequence as set forth in SEQ ID NO: 2, and 50 μl aliquot of transformed cells were spread on an agar plate containing 100 μg/ml Ampicilline. The agar plates were incubated overnight at 37° C. A single colony from the plate was inoculated in 10 ml LB medium in a flask containing 100 μg/ml Ampicilline. The culture was grown overnight at 37° C. with shaking at 250 rpm.

Similarly, the nucleotide sequence as set forth in SEQ ID NO: 8 and SEQ ID NO: 12 were cloned separately in pET-32a(+) expression vector to obtain recombinant vectors comprising the said 1325 nucleotide sequence and subsequently E. coli cells (BL21(DE3)) were transformed separately with these recombinant vectors to obtain recombinant E. coli cells.

### Example 3: Expression of Vip Proteins

The culture from Example 2 (Vip Protein-1, Vip Protein-2, and Vip Protein-3) was sub-cultured by inoculation of 10 ml of the LB medium in a 250 ml flask with 0.1 ml from overnight grown culture. The cells were grown at 37° C. with shaking at 250 rpm until the OD600 reached 0.5-0.6. IPTG (1 mM final concentration) was added and the culture was incubated at 30° C. with shaking at 150 rpm. The overnight grown culture was centrifuged for 10 min at 6,000 rpm and cells were collected at 4° C. The cells were re-suspended in 0.4 ml lysis buffer and the suspension was transferred to a 1.5 ml micro centrifuge tube. The tube was briefly vortexed for 5-10 minutes to thoroughly re-suspend the cells and to ensure full extraction of the soluble protein. The cell lysate was centrifuged at 14000 rpm for 5 min to pellet any insoluble material. The soluble protein fraction was carefully removed from the cell debris. The clear supernatant was analyzed by SDS-PAGE to assess isolation efficiency and identity of the Vip proteins.

### Example 4: Purification of Vip Proteins

The recombinant Vip proteins obtained in Example 3 was purified by column filtration using kit from BioRad, USA. For purification of the recombinant Vip protein, required amount Ni-NTA Agarose was transferred into the column and storage buffer was drained out by gravity flow. The column was equilibrated by adding lysis buffer and excess lysis buffer was drained by gravity flow. The soluble protein fraction was loaded on to the column and the flow through was collected. Wash buffer was added to the column and allowed it to drain out by gravity flow. Washing of column was repeated twice. Elution buffer containing required amount of Imidazole was used to drain out column and collected the elute containing His-tagged recombinant Vip protein. The eluted protein was dialysed to remove Imidazol and obtain pure protein.

### Example 5: Insect Diet Bioasy

Bioassay with recombinant Vip proteins (SEQ ID NO: 1; SEQ ID NO: 7& SEQ ID NO: 11) To determine the toxicity of the Vip proteins insect diet bioassay was conducted against the first-instar larvae of Spodoptera frugiperda and Helicoverpa armigera by using diet incorporation method.

### Artificial Diet Preparation

All the components as mentioned in Table 1 (except dried yeast and agar) were weighed and mixed in a large bowl containing 500 ml of preboiled warm water. Fifty-three grams of active dried yeast was mixed with 350 ml of water in a separate beaker. Agar (16 g) was dissolved in 350 ml of water and boiled for 5 min. The yeast and agar suspensions were transferred to the bowl containing remaining diet components which was prepared earlier. The slurry was boiled for 5 min and then blended thoroughly. To this slurry, 10% formaldehyde (13.5 ml) was added and mixed with a spatula. The slurry was then cooled to 50° C.

TABLE 1
Composition of artificial diet

| S. No. | Component | Quantity |
|---|---|---|
| 1 | Chickpea flour | 160.0 g |
| 2 | Methyl Para-hydroxy benzoate | 3.3 g |
| 3 | Baker's yeast | 53.0 g |
| 4 | Ascorbic acid | 5.3 g |
| 5 | Agar-agar | 16.0 g |
| 6 | Sorbic acid | 1.7 g |
| 7 | Streptomycin sulphate | 0.25 g |
| 8 | Multivitamin capsule | 2.0 Nos. |
| 9 | Formalin (10% solution) | 13.5 ml |
| 10 | Distilled water | 1200 ml |

Three artificial diets each containing one type of purified recombinant vip protein i.e. Vip protein-1, Vip protein-2 and Vip protein-3 at different concentrations (10 μg/ml, 15 μg/ml, 20 μg/ml and 25 μg/ml) were prepared by adding the said Vip protein separately in the lukewarm artificial diet (approx 50° C.) and mixing thoroughly. A negative control (without any Vip protein) was also prepared where the diet was mixed with the buffer. A positive control diet containing Vip protein (Accession no. DQ539887.1) at different concentrations (10 μg/ml, 15 μg/ml, 20 μg/ml and 25 μg/ml) was prepared. Subsequently, the diet was poured on to the petriplates and the plates were exposed to UV light in the laminar airflow for 1 h to surface sterilize the diet. The diet was cut into equal-sized pieces and placed in sterile labelled cups.

The first-instar larvae were tested for the toxicity of the Vip proteins. The insects were released on the artificial diets containing the recombinant Vip proteins, a positive control and negative control diets. one larva was placed in each cup and the lid was closed. Each bioassay was repeated 5 times, with one first-instar larva per replication for each concentration. The insects were incubated at 28±2° C., 65±5% relative humidity, and 14:10 L:D photoperiod. The observations were recorded after 7 days.

After 7 days lethal concentrations (LC50) value was estimated using the standard statistical analysis method and LC (50) of the insects was observed at a concentration ranging from 5 μg/ml to 6.5 μg/ml of the recombinant Vip protein. Surprisingly, it was observed that all the three recombinant Vip proteins(Vip protein-1, Vip protein-2 and Vip protein-3 separately) shows the similar LC (50) value ranging from 5 μg/ml to 6.5 μg/ml while the LC (50) value the positive control Vip protein (Accession no. DQ539887.1) was observed at 8.23 μg/ml.

### Example 6: Construction of Plant Transformation Vector

The pUC57 vector carrying the DNA sequence as set forth in SEQ ID NO: 5 encoding Vip protein-1 was digested with restriction enzymes to release the DNA fragment of Vip protein-1 (Reaction volume-20 μl, Plasmid DNA-8.0 μl, 10× buffer-2.0 μl, Restriction enzyme Bam HI and Eco RI-0.5 μl, Distilled water-9.5 μl). All the reagents were mixed and the mixture was incubated at 37° C. for 30 mins. The product obtained after the restriction digestion was analysed by gel electrophoresis and further purified further.

The released fragment was sub-cloned into pUC57 carrying CaMv 35S cassette. The ligation was carried out using T4DNA ligase enzyme (Reaction volume-30 μl, 10× ligation buffer-3.0 μl, Vector DNA-5.0 μl, Insert DNA-15.0 μl, T4 DNA Ligase enzyme-1.0 μl, Distilled water −6.0 μl). The reagents were mixed well and the resulting reaction mixture was incubated at 16° C. for 2 hours. Subsequently competent cells of E. coli DH5 aplha strain were transformed with the ligation mixture comprising the pUC57 carrying CaMv 35S cassette carrying the DNA sequence as set forth in SEQ ID NO: 5 by adding 10 μl of ligation mixture to 100 μl of E. coli DH5 aplha competent cells. The cell mixture thus obtained was placed on ice for 30 mins and incubated at 42° C. for 60 sec in a water bath for heat shock and placed the cell mixture back to ice for 5-10 mins. Subsequently 1 ml LB broth was added to the mixture and further incubated at 37° C. for 1 hour in incubator shaker at 200 rpm. The cell suspension (100 μl) was uniformly spread on LB agar medium containing 30 μg/ml Kanamycin. The plates were incubated at 37° C. overnight. The positive clones were confirmed by restriction digestion analysis. Plasmid was isolated from the positive clones and digested with Eco RV to release the fragment for cloning in pGreen0029.

The Ti plasmid pGreen0029 vector was prepared by restriction digestion with EcoRV enzyme (Reaction volume-20 μl, Plasmid DNA-8.0 μl, 10× buffer-2.0 μl, Restriction enzyme EcoRV-0.5 μl, Distilled water-9.5 μl). All the reagents were mixed and the mixture was incubated at 37° C. for 30 mins. The product obtained after the restriction digestion was analysed by gel electrophoresis.

The purified Vip protein-1 DNA fragment (SEQ ID NO: 5) was ligated in linearized pGreen0029 vector. The ligation was carried out using T4DNA ligase enzyme (Reaction volume-30 μl, 10× ligation buffer-3.0 μl, Vector DNA-5.0 μl, Insert DNA-15.0 μl, T4 DNA Ligase enzyme-1.0 μl, Distilled water −6.0 μl). The reagents were mixed well and the resulting reaction mixture was incubated at 16° C. for 2 hours. Subsequently competent cells of E. coli DH5 aplha strain were transformed with the ligation mixture comprising the pGreen0029 vector carrying the DNA sequence (SEQ ID NO: 5) by adding 10 μl of ligation mixture to 100 μl of E. coli DH5 aplha competent cells. The cell mixture thus obtained was placed on ice for 30 mins and incubated at 42° C. for 60 sec in a water bath for heat shock and placed the cell mixture back to ice for 5-10 mins. Subsequently 1 ml LB broth was added to the mixture and further incubated at 37° C. for 1 hour in incubator shaker at 200 rpm. The cell suspension (100 μl) was uniformly spread on LB agar medium containing 30 μg/ml kanamycin. The plates were incubated at 37° C. overnight. The positive clones were confirmed by restriction digestion analysis. The recombinant vector thus obtained was designated as pGreen0029 CaMV35S-Vip proetin-1.

Thus, the recombinant plasmid pGreen0029-CaMV35S-Vip proetin-1 contains the plant-optimized Vip proetin-1 DNA sequence (SEQ ID NO: 5) under the transcriptional control of the 35SCaMV promoter. Further, pGreen0029-CaMV35S-Vip proetin-1 contains nptll gene, a plant selectable marker gene under the transcriptional control of NOS promoter. The physical arrangement of the components of the pGreen0029-CaMV35S-Vip proetin-1 T-region is illustrated as:

RB>35SCaMV:Vip proetin-1 CDS: CaMV polyA>NOS promoter: npt//CDS: NOS Poly A>LB

Subsequently, recombinant pGreen0029 vectors carrying the DNA sequences as set forth in SEQ ID NO: 6, SEQ ID NO: 9, SEQ ID NO: 10, 1 SEQ ID NO: 3 and SEQ ID NO: 14 were constructed as described above.

### Example 6 (a): Transformation of Agrobacterium Tumefaciens

Agrobacterium tumefaciens strain LBA4404 was transformed with the recombinant pGreen0029-CaMV35S vector(s) obtained from Example 6. 200 ng of the plasmid DNA was added to an aliquot of 100 μl of A. tumefaciens strain LBA4404 competent cells. The mixture was incubated on ice for 30 min and transferred to liquid nitrogen for 20 mins followed by thawing at room temperature. The Agrobacterium cells were then transferred to 1 ml LB broth and incubated at 28° C. for 24 hours in water bath shaker at 200 rpm. The cell suspension was uniformly spread on LB agar medium containing 50 μg/ml rifampicin, 30 μg/ml kanamycin and 5 μg/ml tetracycline. The plates were incubated at 28° C. overnight. Transformed Agrobacterium cells were analysed plasmid extraction and restriction digestion method and positive A. tumefaciens colonies were selected and stored for further use.

### Example 7: Agrobacterium-Mediated Tobacco Transformation

Experimental details of tobacco transformation are described below. Those skilled in the art of tobacco transformation will understand that other methods are available for cotton transformation and for selection of transformed plants when other plant expressible selectable marker genes are used.

### Material

### Plant Material

Tobacco (Nicotiana tabacum): Accession No: P1105952
   Acrobacterium-mediated transformation of tobacco leaf discs:

Tobacco seeds were sterilized using 25% (v/v) bleach+0.1% (v/v) Tween-20 for 20 minutes and washed the seeds with sterile water 5 times. The seeds were then inoculated on MS (Murashige and Skoog) medium for germination.

Leaf explants form 4-6 weeks old tobacco plants were prepared (0.5 cm²) from healthy green leaves and pre-cultured with abaxial surface down on tobacco regeneration medium (TRM) comprising MS medium, BAP 2 mg/L and NAA 1 mg/L and were incubated at 25° C. for overnight to three days under 16:8 light:dark photoperiod.

Transformed Agrobacterium tumefaciens comprising the recombinant plasmid pGreen0029-CaMV35S-Vip proetin-1 were grown overnight at 28° C. at 250 rpm. The overnight grown culture was centrifuged and re-suspended in liquid ½MS medium for final OD₆₀₀ 0.5. The pre-cultured tobacco leaf explants were then co-cultivated with the Agrobacterium suspension for 30 min at room temp with occasional gentle shaking. Excess Agrobacterium were then removed by placing the explants on sterile filter paper and the infected explants were the co-cultivated keeping abaxial surface touching the medium (TRM) at 25° C. under 16:8 light:dark photoperiod for 2-3 days. The explants were then washed and cultured on place them on TRM containing 300 mg/L Timentin and 100 mg/L Kanamycin. The explants were sub-cultured biweekly until shoot emerges out. Regenerated shoots were then transferred to MS medium containing 300 mg/L Timentin and 100 mg/L Kanamycin for rooting. The rooted tobacco plantlets were then transferred to pots under controlled conditions. The putative transgenic tobacco plants were confirmed by the standard PCR analysis method known in the art. The PCR positive plants were then utilized for detached leaf bioassay.

Those skilled in the art of tobacco transformation and regeneration will understand that other methods are available for tobacco transformation, regeneration. Also for selection of transformed plants other plant expressible selectable marker genes can be used.

### Example: 8 Leaf Based Bioassy

### Detached Leaf Bioassay

Detached leaf bioassay against Spodoptera litura was carried out using transgenic tobacco plants expressing individually Vip protein-1, Vip protein-2, and Vip protein-3 and non transgenic (control) tobacco leaf samples. Detached leaf of transgenic tobacco plants expressing the Vip proteins of the invention was plated onto perti plates and then inoculated with single first-instar larva of Spodoptera litura The assays were performed in triplicate. After seven days it was observed that the leaf area damage was almost nil in transgenic tobacco leaf in comparison to the non-transformed control plant (FIG. 1).

Although the foregoing invention has been described in some detail by way of illustration and example for purposes of clarity of understanding, it will be obvious that certain changes and modifications may be practiced within the scope of the present disclosure.

1. An insecticidal protein having an amino acid sequence selected from the group consisting of SEQ ID NO: 1, SEQ ID NO: 7 and SEQ ID NO: 11, when expressed in a plant, results in an insecticidal activity.

2. A recombinant nucleic acid molecule comprising a nucleotide sequence encoding the protein as claimed in claim 1.

3. The recombinant nucleic acid molecule as claimed in claim 2, wherein said nucleic acid molecule comprises the nucleotide sequence selected from the group consisting of SEQ ID NO: 2, SEQ ID NO: 5, SEQ ID NO: 6, SEQ ID NO: 8, SEQ ID NO: 9, SEQ ID NO: 10, SEQ ID NO: 12, SEQ ID NO: 13 and SEQ ID NO: 14.

4. The recombinant nucleic acid molecule as claimed in claim 2, wherein said nucleic acid molecule comprises the nucleotide sequence selected from the group consisting of SEQ ID NO: 5, SEQ ID NO: 6, SEQ ID NO: 9, SEQ ID NO: 10, SEQ ID NO: 13 and SEQ ID NO: 14.

5. The recombinant nucleic acid molecule as claimed in claim 4, wherein the nucleotide sequence is a synthetic sequence which has been designed for optimum expression in a plant.

6. The recombinant nucleic acid molecule as claimed in claim 2, wherein the nucleic acid molecule comprises a promoter operably linked to the nucleotide sequence encoding the protein having an amino acid sequence selected from the group consisting of SEQ ID NO: 1, SEQ ID NO: 7 and SEQ ID NO: 11.

7. An expression cassette comprising a heterologous promoter operably linked to the nucleotide sequence as claimed in claim 2.

8. The expression cassette as claimed in claim 7, wherein said promoter functions in plants is selected from the group consisting of inducible, constitutive, tissue-preferred and tissue-specific promoters.

9. A vector comprising the recombinant nucleic acid molecule as claimed in claim 1.

10. A host cell comprising: the protein as claimed in claim 1, the recombinant nucleic acid molecule as claimed in claim 1 or a vector as claimed in claim 9.

11. The host cell as claimed in claim 10, wherein the host cell is a bacterium, cynobacterium, virus, fungi, insect, or yeast.

12. A method for producing a plant exhibiting an insecticidal activity, wherein said method comprises at least the step of introducing the nucleic acid molecule as claimed in claim 1.

13. The method as claimed in claim 12, wherein said introducing is performed by introgressing into said plant a nucleic acid molecule encoding the protein having the amino acid sequence selected from the group consisting of SEQ ID NO: 1, SEQ ID NO: 7 and SEQ ID NO: 11.

14. The method as claimed in claim 13, wherein said introducing is performed by;
(a) inserting the recombinant nucleic acid molecule as claimed in claim 2 into a plant cell, wherein said nucleic acid molecule further comprises a promoter functional in a plant cell and a terminator, wherein said promoter and terminator is operably linked to the nucleotide sequence encoding the protein having the amino acid sequence selected from the group consisting of SEQ ID NO: 1, SEQ ID NO: 7 and SEQ ID NO: 11,
(b) obtaining a transformed plant cell from said plant cell of step (a), wherein said transformed plant cell comprises the nucleotide sequence encoding at least one said protein; and
(c) generating a transgenic plant from said transformed plant cell of step (b), wherein said transgenic plant comprises the nucleotide sequence encoding at least one said protein.

15. A transgenic plant comprising a recombinant nucleic acid molecule comprising a nucleotide sequence encoding the protein as claimed in claim 1, wherein said plant exhibits an insecticidal activity.

16. A recombinant microorganism comprising a recombinant nucleotide sequence which encodes the insecticidal protein as claimed in claim 1.

17. The recombinant microorganism as claimed in claim 16, wherein said nucleotide sequence is selected from the group consisting of SEQ ID NO: 2, SEQ ID NO: 5, SEQ ID NO: 6, SEQ ID NO: 8, SEQ ID NO: 9, SEQ ID NO: 10, SEQ ID NO: 12, SEQ ID NO: 13 and SEQ ID NO: 14.

18. The recombinant microorganism as claimed in claim 16, wherein said microorganism is selected from the group consisting of bacteria, virus, algae and fungi.

19. The recombinant microorganism as claimed in claim 16 further comprising a second nucleotide sequence which encodes a second insecticidal protein.

20. The recombinant microorganism as claimed in claim 19, wherein said second nucleotide sequence encodes a delta endotoxin or a vegetative insecticidal protein.

21. A composition comprising the recombinant microorganisms as claimed in claim 16.

22. Use of the insecticidal protein as claimed in claim 1 or the recombinant nucleic acid molecule as claimed in 2 for production of insecticidal composition.

23. Use of the recombinant nucleic acid molecule as claimed in claim 2, the expression cassette as claimed in claim 7 or the vector as claimed in claim 9 for production of insect resistant transgenic plants.

24. Use of the recombinant microorganism as claimed in claim 16 or the composition as claimed in claim 21 for the control of insect pests.


SEQUENCE LISTING (14 sequences)

SEQ ID NO: 1 - protein, 789 aa, synthetic construct
MNMNNAKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKPDAINTMLRVYLPKITSMLSDVMKQNYALSLQIEYLSKQLQEISDKLDIINVNVLINSTLTEITPAYQRIKYVNEKFEELTFATETSSKVEKDGSPADILDELTELTELAESVTKNDVDGFEFYLNTFHDVMVGNNLFGRSALKTATELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIFGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNVDEAWGDSFIILEISPSEKLLSPELINTNNWTSTGSTNISGNTLTLYQGGRGILKQNLQLDSFSTYRVSFSVSGDANVRIRNSREVLFEKRYMSGAKDVSEMFTTKFEKDNFYIELSQGNNLYGGPIVHFYDVSIR

SEQ ID NO: 2 - DNA, 2370 bp, synthetic construct
  source 1..2370: mol_type = other DNA
atgaatatgaataatgctaaattaagcacacgagccttaccaagttttattgattattttaatggcatttatggatttgccactggtatcaaagacattatgaacatgatttttaaaacggatacaggtggtgatctaaccctagacgaaattttaaagaatcagcagttactaaatgatatttctggtaaattggatggggtgaatggaagcttaaatgatcttatcgcacagggaaacttaaatacagaattatctaaggaaatattaaaaattgcaaatgaacaaaatcaagttttaaatgatgttaataacaaacccgatgcgataaatacgatgcttcgggtatatctacctaaaattacctctatgttgagtgatgtaatgaaacaaaattatgcgctaagtctgcaaatagaatacttaagtaaacaattgcaagagatttctgataagttggatattattaatgtaaatgtacttattaactctacacttactgaaattacacctgcgtatcaaaggattaaatatgtgaacgaaaaatttgaggaattaacttttgctacagaaactagttcaaaagtagaaaaggatggctctcctgcagatattcttgatgagttaactgagttaactgaactagcggaaagtgtaacaaaaaatgatgtggatggttttgaattttaccttaatacattccacgatgtaatggtaggaaataatttattcgggcgttcagctttaaaaactgcaacggaattaattactaaagaaaatgtgaaaacaagtggcagtgaggtcggaaatgtttataacttcttaattgtattaacagctctgcaagcaaaagcttttcttactttaacaacatgccgaaaattattagggttagcagatattgattatacttctattatgaatgaacatttaaataaggaaaaagaggaatttagagtaaacatcctccctacactttctaatactttttctaatcctaattatgcaaaagttaaaggaagtgatgaagatgcaaagatgattgtggaagctaaaccaggacatgcattgattgggtttgaaattagtaatgattcaattacagtattaaaagtatatgaggctaagctaaaacaaaattatcaagtcgataaggattccttatcggaagttatttttggtgatatggataaattattgtgcccagatcaatctgaacaaatctattatacaaataacatagtatttccaaatgaatatgtaattactaagattgatttcactaaaaaaatgaaaactttaagatatgaggtaacagcgaatttttatgattcttctacaggagaaattgacttaaataagaaaaaagtagaatcaagtgaagcggagtatagaacgttaagtgctaatgatgatggggtgtatatgccgttaggtgtcatcagtgaaacatttttgactccgattaatgggtttggcctccaagctgatgaaaattcaagattaattactttaacatgtaaatcatatttaagagaactactgctagcaacagacttaagcaataaagaaactaaattgatcgtcccgccaagtggttttattagcaatattgtagagaacgggtccatagaagaggacaatttagagccgtggaaagcaaataataagaatgcgtatgtagatcatacaggcggagtgaatggaactaaagctttatatgttcataaggacggaggaatttcacaatttattggagataagttaaaaccgaaaactgagtatgtaatccaatatactgttaaaggaaaaccttctattcatttaaaagatgaaaatactggatatattcattatgaagatacaaataataatttagaagattatcaaactattaataaacgttttactacaggaactgatttaaagggagtgtatttaattttaaaaagtcaaaatgtagatgaagcttggggagatagctttattattttggaaattagtccttctgaaaagttattaagtccagaattaattaatacaaataattggacgagtacgggatcaactaatattagcggtaatacactcactctttatcagggaggacgagggattctaaaacaaaaccttcaattggatagtttttcaacttatagagtgtctttttctgtgtccggggatgctaatgtaaggattagaaattctagggaagtgttatttgaaaaaagatatatgagcggtgctaaagatgtttctgaaatgttcactacaaaatttgagaaagataacttttatatagagctttctcaagggaataatttatatggtggtcctattgtacatttttacgatgtctctattaggtag

SEQ ID NO: 3 - DNA, 25 bp, synthetic construct
  source 1..25: mol_type = other DNA
atgamyawgaataatrctaarttaa

SEQ ID NO: 4 - DNA, 24 bp, synthetic construct
  source 1..24: mol_type = other DNA
ytahytaatagagamatcrtaaaa

SEQ ID NO: 5 - DNA, 2370 bp, synthetic construct
  source 1..2370: mol_type = other DNA
atgaatatgaataatgccaagctttctacgagagctcttccatctttcattgactatttcaatggtatctacgggttcgcgaccggcatcaaagatattatgaacatgatcttcaaaacagatacaggtggagatttgacgttagatgagatcctgaaaaatcaacaactcttaaacgatattagcggcaaacttgacggggtaaatggtagtctaaatgatcttatagcacagggtaacctaaatacggagctaagtaaggaaattcttaagattgctaacgagcagaatcaagttttaaatgacgttaacaacaaacctgatgccattaacacaatgctccgagtgtacctccccaaaattacttctatgctttctgatgttatgaaacaaaactatgctttaagtctccaaatagaatacctctccaaacagcttcaagaaatatcagataagctcgatataatcaacgtcaacgtacttattaattcaacgttgactgagatcacgccagcatatcaaagaatcaagtatgttaatgagaaatttgaagaactcacatttgcaactgaaacctcttctaaggtcgagaaggatggttctccagcggacattcttgatgagttgacggagttgacagaactagcagagtcagttactaagaacgacgtcgacggctttgagttttatctcaataccttccacgacgtcatggtgggaaacaatctctttggcaggagcgcgctaaagacagctaccgagctgatcaccaaagagaacgttaaaaccagcggcagtgaggtcggtaacgtatataacttccttatcgttttgacagctttacaagctaaggctttcctcactctcaccacttgcaggaaactgctgggacttgctgatattgactatacaagtatcatgaacgagcatttgaacaaggaaaaagaagagtttagggttaatatactccctacactctctaatacgttttctaatcctaattatgcaaaagttaagggttcagacgaagatgccaagatgatcgtggaagcaaagcctggtcatgcacttataggtttcgaaatcagtaatgatagtattacagtcctaaaggtgtacgaggctaagttaaagcaaaactatcaagttgataaagacagtctttcagaggttatcttcggtgatatggataagttgttgtgtccagatcagtctgaacagatttattatacaaacaatatagtttttcctaacgagtatgttattactaagatcgacttcacaaagaaaatgaagactcttaggtatgaagtgactgctaatttttacgacagtagcaccggagaaatcgaccttaataaaaaaaaggttgaatctagcgaggcggagtacagaactctttcagccaatgatgatggggtgtatatgcctttaggcgtgatctccgaaacttttcttacgccgattaacgggttcgggcttcaagccgatgaaaatagcagattgataacactaacatgcaaatcgtacttgagagaattgttgctagctaccgatttgagcaataaagaaaccaagctgattgtcccaccttctggctttatttcaaatattgtggagaacggatcaatcgaggaagacaaccttgaaccgtggaaggcgaacaacaagaacgcttatgtagaccatactggcggtgttaacggaacaaaagcgctatatgttcataaagatggtggaatcagtcaattcataggggacaaactcaagccaaagacagagtacgttatacagtacacagtcaaaggaaaaccgagcattcatctcaaagatgagaacactggatatattcattacgaggacaccaacaacaatttggaggactaccaaaccataaataagaggtttacaacagggacagatttaaagggagtatatcttatcctcaagagtcaaaatgtggacgaagcttggggtgacagttttatcattttggaaatatccccgagtgagaagttgttgtccccggaactaatcaacactaataattggacatcaacaggctccaccaacatttcgggaaacactttgacattgtaccaaggcggacgaggtattcttaaacaaaatctccagctcgactcattttctacctatagagtgtcattctctgtgtctggagacgctaacgttcgtatccgtaacagtagagaagtattgttcgaaaaaagatatatgtcaggtgccaaagatgtttcggagatgttcacgactaagttcgagaaggacaacttttatatcgagttaagccagggtaataacctgtatggcggacctatcgttcacttttacgatgtaagcattagatga

SEQ ID NO: 6 - DNA, 2370 bp, synthetic construct
  source 1..2370: mol_type = other DNA
atgaatatgaacaacgctaagttgtccacccgggctttgccgagtttcatagactacttcaatgggatctacggatttgccactggcattaaggatattatgaacatgatttttaagacggatacaggcggcgatctgacattggacgagatcctcaagaatcagcagcttctcaacgacatctcgggaaagctggatggcgtgaacggcagcttgaacgatctgatcgcccaggggaatctcaacacggaattgagcaaggagattctgaagatcgcaaacgaacaaaatcaggtcctgaatgatgttaataacaagccggatgccataaacaccatgctgcgggtctacttgccaaagataactagtatgctttccgatgtcatgaagcagaattacgcactgagtttgcaaatcgaatacttgagcaaacagcttcaggagatctcggataagctcgacatcattaacgtgaacgttctcatcaattcgacgctgacagagatcacaccggcataccagcggatcaagtacgttaacgagaagtttgaagagctcactttcgccactgagacctcatctaaggtcgagaaggacggctcgccagcggacatccttgatgaactcacggagctcaccgagctggctgaaagcgtgacgaagaacgacgtcgacggcttcgaattttacctgaatacgtttcacgatgttatggtgggaaacaatctctttggtaggtccgccttgaagaccgccaccgagctcatcacgaaggagaacgttaaaacctccggtagcgaggtcggcaacgtttacaacttcctgatcgtgctgaccgctctgcaggcaaaggcattcctgacacttacaacgtgcaggaagctccttggccttgcggatatagattacacgagtatcatgaatgagcatttgaacaaggagaaagaggagtttagggttaacatcctccccactctctcaaatacattctccaacccaaactatgccaaagtgaaagggtctgacgaggacgctaagatgatagtggaggctaagcccggacatgcgctgataggcttcgagattagcaacgactctataacggtgcttaaagtttacgaggctaaactcaagcagaactatcaggttgacaaggatagtctctccgaggtgatctttggtgacatggacaaactgctttgtcctgatcagtcggaacaaatctactacacaaacaatattgtcttccccaacgagtacgttatcacgaagatcgatttcacaaagaagatgaagacgctgaggtacgaggttactgctaatttctacgactcgtcgactggcgagatcgatttgaacaaaaagaaggtcgagtcttcggaggccgaatataggacattgtccgctaatgacgatggtgtttacatgcccttgggagttataagcgagaccttcctgacaccgataaacggattcggactgcaggccgatgagaacagtcgccttatcacgctcacatgtaagagttacttgcgggaattgctgctggcgaccgacctctcaaacaaagaaacaaaacttattgttcctccaagcggtttcatcagtaacatagtggagaacggtagcatagaggaggataatcttgaaccgtggaaagccaacaacaagaatgcctatgtcgaccatactggtggcgtgaacggaacaaaggctttgtatgttcacaaggacggagggatctctcaatttattggcgacaaactgaagccaaagacggagtacgtcattcagtacaccgttaagggtaaaccctccatacacctcaaggacgagaacacaggttacattcattatgaagatacgaataataacttggaggactaccagaccatcaacaagcggttcacaaccggcaccgatctgaagggcgtgtacttgattttgaagagccaaaatgtggacgaggcatggggagacagcttcatcattctggagatctccccttcggagaagttgctctctccagagctgattaacactaacaactggacctccaccgggagcaccaacatttccgggaacacactgacactctaccaaggtggtcgcggaatcttgaagcagaatcttcagctggactccttctccacataccgcgtctcgttctccgtttctggcgacgcaaatgtcaggatcaggaactctagggaagttctgttcgagaagcgctacatgagcggcgcaaaggacgtcagcgagatgttcacaactaaattcgaaaaggataacttctatatcgagcttagtcagggcaataatctgtacggaggccccattgtgcatttctatgatgtgtcaatcagatga

SEQ ID NO: 7 - protein, 789 aa, synthetic construct
MNMNNTKLSTRALPSFIDYFNGINGFATGIKDIMNMIFKTDTGGDLTLDEILKNQQLLNDISGKLDGVNGSLNDLIAQGNLNTELSKEILKIANEQNQVLNDVNNKPDAINTMLRVYLPKITSMLSDVMKQNYALSLQIEYLSKQLQEISDKLDIINVNVLINSTLTEITPAYQRIKYVNEKFEELTFATETSSKVEKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIFGDMDKLLCPDQSEQIYYTNNIVFPNEYVITKIDFTKKMKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVSFSVSGDANVRIRNSREVLFEKRYMSGAKDVSEMFTTKFEKDNFYIELSQGNNLYGGPIVHFYDVSIR

SEQ ID NO: 8 - DNA, 2370 bp, synthetic construct
  source 1..2370: mol_type = other DNA
atgaatatgaataatactaaattaagcacaagagccttaccaagttttattgattattttaatggcattaatggatttgccactggtatcaaagacattatgaacatgatttttaaaacggatacaggtggtgatctaaccctagacgaaattttaaagaatcagcagttactaaatgatatttctggtaaattggatggggtgaatggaagcttaaatgatcttatcgcacagggaaacttaaatacagaattatctaaggaaatattaaaaattgcaaatgaacaaaatcaagttttaaatgatgttaataacaaacccgatgcgataaatacgatgcttcgggtatatctacctaaaattacctctatgttgagtgatgtaatgaaacaaaattatgcgctaagtctgcaaatagaatacttaagtaaacaattgcaagagatttctgataagttggatattattaatgtaaatgtacttattaactctacacttactgaaattacacctgcgtatcaaaggattaaatatgtgaacgaaaaatttgaggaattaacttttgctacagaaactagttcaaaagtagaaaaggatggctctcctgcagatattcttgatgagttaactgagttaactgaactagcgaaaagtgtaacaaaaaatgatgtggatggttttgaattttaccttaatacattccacgatgtaatggtaggaaataatttattcgggcgttcagctttaaaaactgcatcggaattaattactaaagaaaatgtgaaaacaagtggcagtgaggtcggaaatgtttataacttcttaattgtattaacagctctgcaagcaaaagcttttcttactttaacaacatgccgaaaattattagggttagcagatattgattatacttctattatgaatgaacatttaaataaggaaaaagaggaatttagagtaaacatcctccctacactttctaatactttttctaatcctaattatgcaaaagttaaaggaagtgatgaagatgcaaagatgattgtggaagctaaaccaggacatgcattgattgggtttgaaattagtaatgattcaattacagtattaaaagtatatgaggctaagctaaaacaaaattatcaagtcgataaggattccttatcggaagttatttttggtgatatggataaattattgtgcccagatcaatctgaacaaatctattatacaaataacatagtatttccaaatgaatatgtaattactaagattgatttcactaaaaaaatgaaaactttaagatatgaggtaacagcgaatttttatgattcttctacgggagaaattgacttaaataagaaaaaagtagaatcaagtgaagcggagtatagaacgttaagtgctaatgatgatggggtgtatatgccgttaggtgtcatcagtgaaacatttttgactccgattaatgggtttggcctccaagctgatgagaattcaagattaattactttaacatgtaaatcatatttaagagaactactgctagcaacagacttaagcaataaagaaactaaattgatcgtcccgccaagtggttttattagcaatattgtagagaacgggtccatagaagaggacaatttagagccgtggaaagcaaataataagaatgcgtatgtagatcatacaggcggagtgaatggaactaaagctttatatgttcataaggacggaggaatttcacaatttattggagataagttaaaaccgaaaactgagtatgtaatccaatatactgttaaaggaaaaccttctattcatttaaaagatgaaaatactggatatattcattatgaagatacaaataataatttagaagattatcaaactattaataaacgttttactacaggaactgatttaaagggagtgtatttaattttaaaaagtcaaaatggagatgaagcttggggagataactttattattttggaaattagtccttctgaaaagttattaagtccagaattaattaatacaaataattggacgagtacgggatcaactaatattagcggtaatacactcactctttatcagggaggacgagggattctaaaacaaaaccttcaattggatagtttttcaacttatagagtgtctttttctgtgtccggggatgctaatgtaaggattagaaattctagggaagtgttatttgaaaaaagatatatgagcggtgctaaagatgtttctgaaatgttcactacaaaatttgagaaagataacttttatatagagctttctcaagggaataatttatatggtggtcctattgtacatttttacgatgtctctattaggtag

SEQ ID NO: 9 - DNA, 2370 bp, synthetic construct
  source 1..2370: mol_type = other DNA
atgaacatgaacaacacaaaattatcaacaagagctctgccatcgttcattgactacttcaacgggattaacgggttcgcaactggaattaaagatataatgaatatgatcttcaaaacagatactggaggagatctaacacttgacgagattttgaagaatcaacagctgctgaacgatataagtggcaagctggatggtgtcaacggatcgcttaatgacctcatagcacaaggcaacctcaatacagagctttctaaagaaatcctcaagattgctaatgaacagaatcaagtgttaaatgacgtgaacaataagcctgacgctataaacactatgttacgagtttatttgcctaaaattacatcaatgctgtccgatgtcatgaagcaaaactatgccttatctctgcaaattgagtacttatcaaagcagctacaagaaatctctgataagctggatataattaacgtgaatgtgttgataaattcaacgttgaccgagatcacgccagcgtatcaacgtatcaagtacgttaacgagaaatttgaagaacttacatttgcaactgagacaagctctaaggttgagaaggatgggagccctgctgacatattagatgagttaacggaacttaccgagctcgctaaatctgtgactaagaacgatgtggacggatttgagttttatttgaacacattccacgacgttatggtgggaaataacctctttggtcgttctgctttaaagaccgcttcggaactaatcactaaagagaatgtaaaaacctcaggaagtgaggtcggcaatgtgtataatttcctcattgttttgacagctttacaggctaaggcattcttaaccttaacaacatgtagaaagcttctcgggcttgctgatattgactacacaagcatcatgaacgaacatctcaacaaggaaaaagaggagttcagagtgaacatacttccgactctaagcaacactttttctaacccaaattacgctaaagttaaagggtccgatgaggatgctaaaatgatcgtcgaagccaaacctggacacgcacttatcggcttcgaaatatctaatgacagtattactgtcctgaaagtctacgaagccaaacttaaacaaaactatcaagttgacaaagactcattgtctgaggtgatttttggggatatggataaactcctttgcccggatcaaagcgaacaaatctattatactaataatattgtctttccaaatgaatatgtgattaccaagatcgactttacgaagaaaatgaagacgcttagatatgaggttacagcaaacttttacgatagttctactggggagatagatctaaacaagaagaaggttgaatcctctgaagctgaatacaggaccctatctgcgaatgatgatggggtctatatgcctctgggagttatttcagaaacttttcttactcctattaacggattcggcttgcaagccgatgaaaactctaggttgatcaccctgacttgtaagtcttatcttcgtgaactcttgctggccaccgacctctctaacaaggagacaaaattgatcgtacctccgtcaggattcatttcgaacattgttgaaaacggtagtattgaagaggacaacttggagccctggaaagcaaataataagaacgcctacgttgatcacacaggtggggtcaatggaacgaaagctctgtatgttcataaggacggaggcatctcacaatttatcggggacaagttaaagccgaagacagagtacgttattcaatacactgttaaaggaaagccctctatacatttaaaagacgagaatacagggtacattcattatgaggatacaaacaataatcttgaggattaccagactatcaataagaggtttacgaccggaactgatctaaagggagtttacctcatcctaaaatcgcagaatggtgatgaagcttggggtgataatttcattattttagagatttctccttcggagaaactgctatcgccagaactcataaataccaacaattggacatcaacaggttcgacgaatatttcagggaacacgctcactttataccaaggaggtagaggtatcttgaaacaaaacctccagttggatagctttagtacctacagagtatcttttagtgtatctggagacgccaacgttaggattaggaactcaagggaggtgctgtttgaaaaaagatatatgtctggagcgaaagatgtaagcgaaatgttcacgaccaaatttgaaaaagataacttctacatagaactgtcccaaggtaacaatttatacggaggtcctatcgtgcatttttacgatgtctccattcgatag

SEQ ID NO: 10 - DNA, 2370 bp, synthetic construct
  source 1..2370: mol_type = other DNA
atgaatatgaacaatactaagttgtctacgagagccctcccatctttcatcgattacttcaacggcatcaacggcttcgcgactggtatcaaagacatcatgaacatgatatttaaaaccgatacaggcggggaccttacgctggacgaaatcttgaagaaccagcaacttcttaatgatatttccggcaaactggatggcgttaacggttccctcaacgacctcattgctcaaggcaatctgaacacagaactgtcaaaggagatactcaagatcgcaaatgagcagaaccaggtgctgaatgatgtgaacaataagcctgacgccatcaataccatgttgcgcgtttaccttcccaagatcacatccatgttgtcggatgtcatgaagcaaaactacgccctgagcctccagattgagtatctctctaagcagctccaggagattagtgataagttggatataatcaatgttaatgttctcattaactccacgctgactgagataaccccagcgtaccaacggatcaagtatgtgaacgaaaagttcgaagagctgacctttgccactgagacgtcaagtaaagtggagaaggatggctcccccgccgacatccttgacgagttgaccgagcttacagagctcgcgaagtccgtgacgaagaacgatgttgatgggttcgagttctacctcaataccttccatgatgtgatggtggggaacaacctctttggccgctctgcgcttaagacggctagcgagctcataacgaaggagaatgtgaaaacctcggggtctgaggtgggtaatgtgtacaactttctcatagtccttaccgctcttcaggccaaggctttcctcacgctgacaacctgccgcaagctcttggggctcgctgacatagattacacttctattatgaacgaacatttgaacaaggagaaggaagaatttagggttaatatcctcccgacactctccaatacatttagtaacccaaactatgccaaagttaagggctctgacgaggacgcgaagatgattgtggaggcaaaaccagggcacgcccttatcggcttcgagatttccaatgatagtattactgtgcttaaggtctatgaagcgaaactgaaacaaaattaccaagttgataaagattctctgtctgaagtgatcttcggagatatggataagctgctttgtcctgaccagagtgagcaaatctattacaccaataacattgtgttccctaacgagtacgtcatcactaagattgactttacgaagaaaatgaaaaccctgcgttacgaggtcacggccaatttttatgacagtagcaccggcgaaattgatttgaataagaaaaaagttgagtcctccgaagccgaatacagaacgctgagtgctaacgacgacggcgtgtatatgcccctgggcgtgatcagcgagaccttcttgaccccaatcaatgggttcggtttgcaagcggacgagaactctcggctcattacgctgacgtgcaagtcgtacttgagagaattgctcctcgcgaccgatttgtctaacaaggagaccaagcttattgtccccccgtccggcttcatctctaacatcgttgaaaacggatcgattgaagaggacaaccttgaaccctggaaggctaacaacaagaacgcgtatgtcgatcacacagggggggtcaatggcaccaaagctctgtacgtccataaggacggtggtatttctcaattcatcggagataaactcaagccgaagaccgagtacgtgatacagtatacagtcaagggcaagccctccattcacctgaaagacgaaaacacaggctacattcattacgaggataccaataacaacctcgaagactaccagaccataaacaagcggttcacaaccggaacagacctcaagggtgtctatctgattctcaaatcccaaaacggtgatgaggcgtggggtgacaacttcatcattttggagatctccccgtctgaaaagcttctttccccggagcttattaacaccaacaattggacatcgaccggttcaacaaacatatctggtaacacactgacactctatcaaggaggtcggggtatattgaagcaaaacctgcagttggactccttctccacttacagagtttccttctcagtgtcgggtgacgccaacgtcaggataaggaacagcagagaggtcctcttcgagaagcgttatatgagtggcgcaaaggacgtcagcgaaatgtttacgactaaatttgagaaggacaacttctacatcgagcttagccaaggcaataacctgtacggggggccaattgtgcatttctacgacgtctcgattcggtag

SEQ ID NO: 11 - protein, 789 aa, synthetic construct
MNKNNTKLSTRALPSFIDYFNGIYGFATGIKDIMNMIFKTDTGGDLTLDEILKNQQLLNDISGKLDGVNGSLNDLIAQGNLNTELSKEILKIANEQNQVLNDVNNKLDAINTMLRVYLPKITSMLSDVMKQNYALSLQIEYLSKQLQEISDKLDIINVNVLINSTLTEITPAYQRIKYVNEKFEELTFATETSSKVKKDGSPADILDELTELTELAKSVTKNDVDGFEFYLNTFHDVMVGNNLFGRSALKTASELITKENVKTSGSEVGNVYNFLIVLTALQAKAFLTLTTCRKLLGLADIDYTSIMNEHLNKEKEEFRVNILPTLSNTFSNPNYAKVKGSDEDAKMIVEAKPGHALIGFEISNDSITVLKVYEAKLKQNYQVDKDSLSEVIYGDMDKLLCPDQSEQIYYTNNIVFPNEYVITKIDFTKKRKTLRYEVTANFYDSSTGEIDLNKKKVESSEAEYRTLSANDDGVYMPLGVISETFLTPINGFGLQADENSRLITLTCKSYLRELLLATDLSNKETKLIVPPSGFISNIVENGSIEEDNLEPWKANNKNAYVDHTGGVNGTKALYVHKDGGISQFIGDKLKPKTEYVIQYTVKGKPSIHLKDENTGYIHYEDTNNNLEDYQTINKRFTTGTDLKGVYLILKSQNGDEAWGDNFIILEISPSEKLLSPELINTNNWTSTGSTNISGNTLTLYQGGRGILKQNLQLDSFSTYRVYFSVSGDANVRIRNSREVLFEKRYMSGAKDVSEMFTTKFEKDNFYIELSQGNNLYGGPIVHFYDVSIK

SEQ ID NO: 12 - DNA, 2370 bp, synthetic construct
  source 1..2370: mol_type = other DNA
atgaacaagaataatactaaattaagcacaagagccttaccaagttttattgattattttaatggcatttatggatttgccactggtatcaaagacattatgaacatgatttttaaaacggatacaggtggtgatctaaccctagacgaaattttaaagaatcagcagttactaaatgatatttctggtaaattggatggggtgaatggaagcttaaatgatcttatcgcacagggaaacttaaatacagaattatctaaggaaatattaaaaattgcaaatgaacaaaatcaagttttaaatgatgttaataacaaactcgatgcgataaatacgatgcttcgggtatatctacctaaaattacctctatgttgagtgatgtaatgaaacaaaattatgcgctaagtctgcaaatagaatacttaagtaaacaattgcaagagatttctgataagttggatattattaatgtaaatgtacttattaactctacacttactgaaattacacctgcgtatcaaaggattaaatatgtgaacgaaaaatttgaggaattaacttttgctacagaaactagttcaaaagtaaaaaaggatggctctcctgcagatattcttgatgagttaactgagttaactgaactagcgaaaagtgtaacaaaaaatgatgtggatggttttgaattttaccttaatacattccacgatgtaatggtaggaaataatttattcgggcgttcagctttaaaaactgcatcggaattaattactaaagaaaatgtgaaaacaagtggcagtgaggtcggaaatgtttataacttcttaattgtattaacagctctgcaagcaaaagcttttcttactttaacaacatgccgaaaattattagggttagcagatattgattatacttctattatgaatgaacatttaaataaggaaaaagaggaatttagagtaaacatcctccctacactttctaatactttttctaatcctaattatgcaaaagttaaaggaagtgatgaagatgcaaagatgattgtggaagctaaaccaggacatgcattgattgggtttgaaattagtaatgattcaattacagtattaaaagtatatgaggctaagctaaaacaaaattatcaagtcgataaggattccttatcggaagttatttatggtgatatggataaattattgtgcccagatcaatctgaacaaatctattatacaaataacatagtatttccaaatgaatatgtaattactaaaattgatttcactaaaaaaaggaaaactttaagatatgaggtaacagcgaatttttatgattcttctacaggagaaattgacttaaataagaaaaaagtagaatcaagtgaagcggagtatagaacgttaagtgctaatgatgatggggtgtatatgccgttaggtgtcatcagtgaaacatttttgactccgattaatgggtttggcctccaagctgatgaaaattcaagattaattactttaacatgtaaatcatatttaagagaactactgctagcaacagacttaagcaataaagaaactaaattgatcgtcccgccaagtggttttattagcaatattgtagagaacgggtccatagaagaggacaatttagagccgtggaaagcaaataataagaatgcgtatgtagatcatacaggcggagtgaatggaactaaagctttatatgttcataaggacggaggaatttcacaatttattggagataagttaaaaccgaaaactgagtatgtaatccaatatactgttaaaggaaaaccttctattcatttaaaagatgaaaatactggatatattcattatgaagatacaaataataatttagaagattatcaaactattaataaacgttttactacaggaactgatttaaagggagtgtatttaattttaaaaagtcaaaatggagatgaagcttggggagataactttattattttggaaattagtccttctgaaaagttattaagtccagaattaattaatacaaataattggacgagtacgggatcaactaatattagcggtaatacactcactctttatcagggaggacgagggattctaaaacaaaaccttcaattagatagtttttcaacttatagagtgtatttttctgtgtccggagatgctaatgtaaggattagaaattctagggaagtgttatttgaaaaaagatatatgagcggtgctaaagatgtttctgaaatgttcactacaaaatttgagaaagataacttttatatagagctttctcaagggaataatttatatggtggtcctattgtacatttttacgatgtctctattaagtaa

SEQ ID NO: 13 - DNA, 2370 bp, synthetic construct
  source 1..2370: mol_type = other DNA
atgaacaaaaacaatacgaaattatccacgcgagcgctcccgtcattcattgattactttaacggaatctatggatttgctactggtattaaggatataatgaacatgatatttaagacggatacaggaggggacttaactcttgacgagattttaaagaaccaacaactacttaacgatataagtggtaaacttgatggagtcaacggtagtctgaacgaccttattgctcagggtaacctcaacacggaactttctaaagagattttgaaaattgctaacgagcaaaatcaggttctgaatgatgtaaacaataaactcgatgctattaatacaatgttgagagtatacctcccaaaaattacatcaatgctctccgatgtcatgaagcagaactacgcgctctctttacaaatcgaatacctcagcaagcaattgcaagaaattagtgacaagcttgatattattaatgttaacgtccttataaattcgacactaactgaaatcactccagcataccagagaattaaatacgttaatgagaaattcgaagaactaacgttcgctactgagactagttctaaggtaaagaaagatggatctcctgcagatatattggacgaactcaccgaacttaccgagctcgcgaaatcagttacgaagaatgatgttgacggatttgagttttacctaaatacatttcatgatgtcatggtggggaacaacttgttcggtagaagcgccttaaaaaccgcgtctgaacttataacaaaagaaaatgttaaaacctcgggaagtgaggtagggaacgtttacaactttctcatagtgttgactgcattgcaagccaaagcgtttctaaccctcaccacttgtaggaaattgcttggacttgctgatattgactatacttcaatcatgaacgagcaccttaacaaggagaaggaggagttccgagtcaacatccttccaactttgtcaaacacatttagcaacccgaactacgctaaggtcaagggtagtgacgaggatgcaaaaatgattgtcgaggcgaagccgggtcatgcactcatcggattcgaaatatcgaatgattctataacggttttgaaagtctacgaagctaaactcaagcagaattaccaagttgacaaagactctctttctgaggtgatttatggagatatggataagctactttgcccagaccaatctgagcagatttattatacgaataatattgtctttccgaacgagtatgtgatcactaagatagattttaccaagaagaggaagactcttagatatgaggttaccgcaaatttctacgattcgtccaccggtgaaatcgatcttaataaaaaaaaggtcgagtcaagtgaggctgaataccgtactctgagcgcaaacgatgatggggtctacatgcctttgggcgttatttctgaaactttcctgacaccaataaatggctttggcttgcaggctgacgaaaactcacgtttaataactctgacttgtaagagctatcttagggaactcttattggccacggatttatcaaataaagagacgaaactcatcgttcctcccagtggatttatttcaaatattgttgagaatggttccattgaggaggacaaccttgagccatggaaggcgaataacaagaatgcctatgttgatcatactggtggtgttaatggaactaaggcactctatgtgcataaagatggaggtattagtcagtttatcggtgataagctcaagccgaaaacagaatacgtcatccagtatactgtcaaaggcaaaccatcaatacacttaaaggatgaaaacacaggttacatacactatgaggatacaaacaataatcttgaagattatcaaacaattaacaagcgatttactactggtactgatttaaaaggagtatatttgattctaaagagtcaaaacggcgacgaagcctggggtgataattttataatcctagagatctcgccatctgaaaagcttctgtcgccagaacttatcaatacaaacaattggacatctacaggtagtacaaacatctcaggtaacacccttaccctctaccagggaggacgagggattttgaagcaaaaccttcagctggattcatttagcacatatagagtatacttctcagtttctggagacgccaacgttaggattaggaacagtagagaggttctctttgaaaaacgttatatgtcaggtgccaaggatgtttcggagatgttcactactaagttcgaaaaagataacttttacatcgaattaagtcagggcaacaacctttatggtggtcctatagtgcatttctatgacgtttctattaagtag

SEQ ID NO: 14 - DNA, 2370 bp, synthetic construct
  source 1..2370: mol_type = other DNA
atgaacaaaaacaatactaaacttagcaccagagccctgccgagcttcatagattacttcaacggaatctacggcttcgctaccggtatcaaggacatcatgaacatgattttcaagaccgacaccggaggggatctgaccttggatgagatcctgaagaaccaacaacttctcaacgacatctcaggaaagcttgacggggtcaatggcagccttaatgatctgatcgcccagggtaatctgaatactgaactgagcaaggagatcctcaaaattgcaaacgagcagaaccaggtccttaacgacgttaacaataaacttgatgctataaacaccatgcttagggtctatctcccgaagataactagtatgctctcggacgtgatgaagcaaaactatgcgctgagtctccagatcgaatatctcagtaaacaactccaggagattagcgataagctcgacatcataaacgtgaacgttctgattaattccactctgaccgagataacaccggcctatcaaaggatcaagtatgtcaacgagaagttcgaggagctcacctttgccaccgaaacaagttctaaagttaaaaaggacggttccccggctgacatcctggacgaactcactgagctcacggagctcgcaaaatccgtgactaagaacgacgttgatggttttgagttctacctcaatacttttcacgacgtgatggtcggcaacaacctttttggccgtagcgcgcttaagactgcatccgagctcataaccaaggagaacgtcaagacttcgggcagcgaggttggcaacgtctacaactttctcatagtcctgactgccctccaggcgaaggcttttcttacactcactacttgcagaaagctgttgggtctcgctgatatagattataccagcattatgaacgaacatttgaacaaggagaaggaggagttccgcgttaacatcctgccgacgctcagcaacaccttctctaacccgaattatgcgaaggtcaagggctcagacgaggatgccaagatgatcgtcgaagccaagcccggtcatgctctcatagggttcgagatctcaaacgacagtatcaccgtcctcaaggtctacgaggcaaaactgaagcagaactaccaggttgacaaggactcgctgtctgaggtgatatacggtgatatggacaagctcctgtgtcctgatcagagtgagcagatctactacactaataacattgtcttcccgaacgaatacgtgatcaccaaaatcgacttcacaaagaagagaaagacgctgcgctacgaagtcacagcgaacttctacgactcttcgacgggtgaaatcgacctcaataagaagaaggtggaatctagcgaggccgagtatcgtaccttgtcggccaacgatgatggtgtctacatgcccctcggtgttatctccgaaacgttcctcactcccatcaacggctttggcctccaggccgatgaaaactcaaggttgataaccctgacatgcaagagctatttgagagagctgcttctcgcgacggatttgtcgaacaaggagaccaagctgattgtgcctccctccggattcatctcgaatatagtggagaacggttctattgaggaagataacctggagccctggaaggccaacaataagaacgcttacgtggatcacacaggcggcgtcaatggcaccaaggctctgtacgtccacaaggacgggggaatctcccagtttattggtgacaaactcaagcctaagaccgagtatgttatccaatacacggtcaagggcaagccgtccatacacctgaaagacgaaaacacaggctatattcactatgaggatacaaataacaacctggaggactaccaaacgatcaacaaaaggttcaccacggggacagacctcaaaggcgtttacctcatcctcaagtctcaaaacggcgatgaggcctggggagataacttcatcattctggagatctcgccgtccgaaaaactcctgagccccgagcttatcaacacgaataattggacatcgaccggctccaccaacatcagcggcaacacactcactctgtatcaagggggtagaggaattttgaagcagaatctccaacttgatagctttagcacctatcgtgtgtacttcagcgtgtcaggcgacgccaacgttcggattcggaactcgcgcgaggttctcttcgagaagcgctacatgtccggcgccaaggacgtgtccgagatgtttaccactaagttcgaaaaagacaacttctacattgagctgtcccaggggaacaacctctacggcggacctattgtgcacttttatgatgtgtcgatcaaatag